SEQUENCE LISTING (64 sequences)

SEQ ID NO: 1 - DNA, 14 bp, Homo sapiens
  source 1..14: mol_type = genomic DNA
ccacaaggtggcgc

SEQ ID NO: 2 - DNA, 14 bp, Homo sapiens
  source 1..14: mol_type = genomic DNA
ccaccagggggcgg

SEQ ID NO: 3 - DNA, 19 bp, Adeno-associated virus
  source 1..19: mol_type = genomic DNA
ttgcgacaccatgtggtca

SEQ ID NO: 4 - DNA, 20 bp, Rodent protoparvovirus 1
  source 1..20: mol_type = genomic DNA
ctcgcggtagatcactcgtt

SEQ ID NO: 5 - DNA, 21 bp, Rodent protoparvovirus 1
  source 1..21: mol_type = genomic DNA
ccaccactaaatggcattctt

SEQ ID NO: 6 - DNA, 20 bp, Rodent protoparvovirus 1
  source 1..20: mol_type = genomic DNA
ctcgcggtagatcactcgtt

SEQ ID NO: 7 - DNA, 19 bp, H1 parvovirus
  source 1..19: mol_type = genomic DNA
tgctcactagatggcgctc

SEQ ID NO: 8 - DNA, 19 bp, H1 parvovirus
  source 1..19: mol_type = genomic DNA
tgcacagcagaggactctg

SEQ ID NO: 9 - DNA, 19 bp, H1 parvovirus
  source 1..19: mol_type = genomic DNA
agtccaccaagggacggag

SEQ ID NO: 10 - DNA, 19 bp, Mouse papillomavirus
  source 1..19: mol_type = genomic DNA
tgaacagtaggagtcagtt

SEQ ID NO: 11 - DNA, 19 bp, Primate erythroparvovirus 1
  source 1..19: mol_type = genomic DNA
ccaccaatagggggtcata

SEQ ID NO: 12 - DNA, 19 bp, Carnivore protoparvovirus 1
  source 1..19: mol_type = genomic DNA
caaccaggaggtgaaaatc

SEQ ID NO: 13 - DNA, 19 bp, Human betaherpesvirus 5
  source 1..19: mol_type = genomic DNA
cgacccgcacatggcgctg

SEQ ID NO: 14 - DNA, 19 bp, Human alphaherpesvirus 1
  source 1..19: mol_type = genomic DNA
agaccaccaggtggcgcac

SEQ ID NO: 15 - DNA, 19 bp, Human alphaherpesvirus 1
  source 1..19: mol_type = genomic DNA
ccaccggcggggggcggcg

SEQ ID NO: 16 - DNA, 19 bp, Human alphaherpesvirus 1
  source 1..19: mol_type = genomic DNA
cgggcggccgggggcggcg

SEQ ID NO: 17 - DNA, 19 bp, Human gammaherpesvirus 4
  source 1..19: mol_type = genomic DNA
tggccaaaagacggcggtt

SEQ ID NO: 18 - DNA, 19 bp, Human gammaherpesvirus 4
  source 1..19: mol_type = genomic DNA
cacccaacaggtggtgaaa

SEQ ID NO: 19 - DNA, 19 bp, Human gammaherpesvirus 4
  source 1..19: mol_type = genomic DNA
ttggcaccatgtggtgcca

SEQ ID NO: 20 - DNA, 19 bp, Alphapapillomavirus 9
  source 1..19: mol_type = genomic DNA
taaccaccaggtggtgcca

SEQ ID NO: 21 - DNA, 19 bp, Hepatitis B virus
  source 1..19: mol_type = genomic DNA
cgaccaccagttggatcca

SEQ ID NO: 22 - DNA, 19 bp, Hepatitis B virus
  source 1..19: mol_type = genomic DNA
tctacagcatggggcagaa

SEQ ID NO: 23 - skipped

SEQ ID NO: 24 - DNA, 14 bp, synthetic construct
  source 1..14: mol_type = other DNA
ccacnaggtggcag

SEQ ID NO: 25 - DNA, 14 bp, synthetic construct
  source 1..14: mol_type = other DNA
ctgccacctngtgg

SEQ ID NO: 26 - DNA, 14 bp, synthetic construct
  source 1..14: mol_type = other DNA
ggcgggggaccacc

SEQ ID NO: 27 - DNA, 19 bp, synthetic construct
  source 1..19: mol_type = other DNA
aacgctgtggtacaccagt

SEQ ID NO: 28 - DNA, 19 bp, synthetic construct
  source 1..19: mol_type = other DNA
tgaccacatggtgtcgcaa

SEQ ID NO: 29 - DNA, 2716 bp, synthetic construct
  source 1..2716: mol_type = other DNA
  misc_binding 206..224: bound_moiety = CTCF binding sites
ccgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagttgcgacaccatgtggtcactagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgatcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 30 - DNA, 2716 bp, synthetic construct
  source 1..2716: mol_type = other DNA
  misc_binding 2482..2500: bound_moiety = CTCF binding site
ccgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgaactggtgtaccacagcgtttcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 31 - DNA, 2735 bp, synthetic construct
  source 1..2735: mol_type = other DNA
  misc_binding 207..225: bound_moiety = CTCF binding site
ccgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagttgcgacaccatgtggtcactagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgaactggtgtaccacagcgtttcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 32 - DNA, 2735 bp, synthetic construct
  source 1..2735: mol_type = other DNA
  misc_binding 207..225: bound_moiety = CTCF binding site
  misc_binding 2505..2520: bound_moiety = CTCF binding site
ccgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagttgcgacaccatgtggtcactagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgattgcgacaccatgtggtcatcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 33 - DNA, 2887 bp, synthetic construct
  source 1..2887: mol_type = other DNA
  misc_binding 207..225: bound_moiety = CTCF binding site
  misc_binding 283..301: bound_moiety = CTCF binding site
  misc_binding 2578..2596: bound_moiety = CTCF binding site
  misc_binding 2616..2634: bound_moiety = CTCF binding site
  misc_binding 2654..2672: bound_moiety = CTCF binding site
  misc_binding 245..263: bound_moiety = CTCF binding site
ccgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagttgcgacaccatgtggtcattgcgacaccatgtggtcattgcgacaccatgtggtcattgcgacaccatgtggtcattgcgacaccatgtggtcactagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgaactggtgtaccacagcgttactggtgtaccacagcgttactggtgtaccacagcgttactggtgtaccacagcgttactggtgtaccacagcgtttcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 34 - DNA, 2735 bp, synthetic construct
  source 1..2735: mol_type = other DNA
  misc_binding 207..225: bound_moiety = CTCF binding site
  misc_binding 2502..2520: bound_moiety = CTCF binding site
ccgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagttgcgacaccatgtggtcactagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgatgaccacatggtgtcgcaatcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 35 - DNA, 2715 bp, synthetic construct
  source 1..2715: mol_type = other DNA
  misc_binding 211..224: bound_moiety = CTCFbinding site
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagccacaaggtggcgcctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgatcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 36 - DNA, 2715 bp, synthetic construct
  source 1..2715: mol_type = other DNA
  misc_binding 2487..2500: bound_moiety = CTCF binding site
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgaccgccccctggtggtcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 37 - DNA, 2729 bp, synthetic construct
  source 1..2729: mol_type = other DNA
  misc_binding 211..224: bound_moiety = CTCF binding site
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagccacaaggtggcgcctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgaccgccccctggtggtcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 38 - DNA, 2729 bp, synthetic construct
  source 1..2729: mol_type = other DNA
  misc_binding 211..224: bound_moiety = CTCF binding site
  misc_binding 2501..2514: bound_moiety = CTCF binding site
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagccacaaggtggcgcctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgaggtggtcccccgcctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 39 - DNA, 2841 bp, synthetic construct
  source 1..2841: mol_type = other DNA
  misc_binding 211..224: bound_moiety = CTCF binding site
  misc_binding 239..252: bound_moiety = CTCF binding site
  misc_binding 267..280: bound_moiety = CTCF binding site
  misc_binding 2557..2570: bound_moiety = CTCF binding site
  misc_binding 2585..2598: bound_moiety = CTCF binding site
  misc_binding 2613..2626: bound_moiety = CTCF binding site
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagccacaaggtggcgcccacaaggtggcgcccacaaggtggcgcccacaaggtggcgcccacaaggtggcgcctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgaccgccccctggtggccgccccctggtggccgccccctggtggccgccccctggtggccgccccctggtggtcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 40 - DNA, 2730 bp, synthetic construct
  source 1..2730: mol_type = other DNA
  misc_binding 211..224: bound_moiety = CTCF binding site
  misc_binding 2501..2515: bound_moiety = CTCF binding site
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagccacaaggtggcgcctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgaggcggggggaccacctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 41 - DNA, 19 bp, Adeno-associated virus
  source 1..19: mol_type = genomic DNA
actggtgtaccacagcgtt

SEQ ID NO: 42 - DNA, 14 bp, Homo sapiens
  source 1..14: mol_type = genomic DNA
ccgccccctggtgg

SEQ ID NO: 43 - DNA, 14 bp, Homo sapiens
  source 1..14: mol_type = genomic DNA
ggtggtcccccgcc

SEQ ID NO: 44 - DNA, 20 bp, Rodent protoparvovirus 1
  source 1..20: mol_type = genomic DNA
ttgctcactagatggcgctc

SEQ ID NO: 45 - DNA, 21 bp, Rodent protoparvovirus 1
  source 1..21: mol_type = genomic DNA
ttcttacggtaaatcaccacc

SEQ ID NO: 46 - DNA, 21 bp, Rodent protoparvovirus 1
  source 1..21: mol_type = genomic DNA
ttcttacggtaaatcaccacc

SEQ ID NO: 47 - DNA, 19 bp, H1 parvovirus
  source 1..19: mol_type = genomic DNA
gaggcagggaaccacctga

SEQ ID NO: 48 - DNA, 38 bp, H1 parvovirus
  source 1..38: mol_type = genomic DNA
agtccaccaagggacggaggaggcagggaaccacctga

SEQ ID NO: 49 - DNA, 19 bp, H1 parvovirus
  source 1..19: mol_type = genomic DNA
gaggcagggaaccacctga

SEQ ID NO: 50 - DNA, 19 bp, Hepatitis B virus
  source 1..19: mol_type = genomic DNA
gaggcagggaaccacctga

SEQ ID NO: 51 - DNA, 57 bp, Hepatitis B virus
  source 1..57: mol_type = genomic DNA
acctaggttgaccaccagcacctaggttgaccaccagcacctaggttgaccaccagc

SEQ ID NO: 52 - DNA, 38 bp, Hepatitis B virus
  source 1..38: mol_type = genomic DNA
acctaggttgaccaccagccgaccaccagttggatcca

SEQ ID NO: 53 - DNA, 76 bp, Hepatitis B virus
  source 1..76: mol_type = genomic DNA
acctaggttgaccaccagccgaccaccagttggatccacgaccaccagttggatccacgaccaccagttggatcca

SEQ ID NO: 54 - DNA, 95 bp, Human gammaherpesvirus 4
  source 1..95: mol_type = genomic DNA
cacccaacaggtggtgaaacacccaacaggtggtgaaacacccaacaggtggtgaaacacccaacaggtggtgaaacacccaacaggtggtgaaa

SEQ ID NO: 55 - DNA, 114 bp, Human gammaherpesvirus 4
  source 1..114: mol_type = genomic DNA
aaagtggtggacaacccacaaagtggtggacaacccacaaagtggtggacaacccacaaagtggtggacaacccacaaagtggtggacaacccacaaagtggtggacaacccac

SEQ ID NO: 56 - DNA, 19 bp, Human gammaherpesvirus 4
  source 1..19: mol_type = genomic DNA
accgtggtgtaccacggtt

SEQ ID NO: 57 - DNA, 38 bp, Human gammaherpesvirus 4
  source 1..38: mol_type = genomic DNA
ttggcaccatgtggtgccattggcaccatgtggtgcca

SEQ ID NO: 58 - DNA, 19 bp, Carnivore protoparvovirus 1
  source 1..19: mol_type = genomic DNA
ctaaaagtggaggaccaac

SEQ ID NO: 59 - DNA, 38 bp, Carnivore protoparvovirus 1
  source 1..38: mol_type = genomic DNA
caaccaggaggtgaaaatccaaccaggaggtgaaaatc

SEQ ID NO: 60 - DNA, 38 bp, Human alphaherpesvirus 1
  source 1..38: mol_type = genomic DNA
ccaccggcggggggcggcgccaccggcggggggcggcg

SEQ ID NO: 61 - DNA, 57 bp, Human alphaherpesvirus 1
  source 1..57: mol_type = genomic DNA
ccaccggcggggggcggcgccaccggcggggggcggcgccaccggcggggggcggcg

SEQ ID NO: 62 - DNA, 19 bp, Primate erythroparvovirus 1
  source 1..19: mol_type = genomic DNA
atactgggggataaccacc

SEQ ID NO: 63 - DNA, 38 bp, Primate erythroparvovirus 1
  source 1..38: mol_type = genomic DNA
atactgggggataaccaccatactgggggataaccacc

SEQ ID NO: 64 - DNA, 38 bp, Alphapapillomavirus 9
  source 1..38: mol_type = genomic DNA
accgtggtggaccaccaataccgtggtggaccaccaat

(19) United States
(12) Patent Application Publication
(10) Pub. No.: US 2024/0052368 A1
(43) Pub. Date: Feb. 15, 2024

(54) GENERATION OF NEXT GENERATION RECOMBINANT AAV GENE THERAPY VECTORS THAT ADOPT 3D CONFORMATION

(71) Applicant: WISCONSIN ALUMNI RESEARCH FOUNDATION, Madison, WI (US)

(72) Inventors: Kinjal Majumder, Madison, WI (US); Clairine Larsen, Madison, WI (US)

(21) Appl. No.: 18/332,380

(22) Filed: Jun. 9, 2023

Related U.S. Application Data
(60) Provisional application No. 63/350,769, filed on Jun. 9, 2022.

(51) Int. Cl. C12N 15/86

(52) U.S. Cl. CPC C12N 15/86; C12N 2750/14143; C12N 2750/14152; A61K 48/00

(57) ABSTRACT

The present invention provides constructs for producing modified recombinant adeno-associated virus (rAAV) vectors. The constructs comprise one or more CCCTC-binding factor (CTCF) binding sites, which facilitate DNA looping and promote efficient transgene expression. Also provided are modified rAAV virus particles comprising these constructs, methods for producing the modified rAAV virus particles, and methods of using the modified rAAV virus particles to deliver a transgene to a subject.

### CROSS-REFERENCE TO RELATED APPLICATIONS

This application claims priority to U.S. Provisional Application No. 63/350,769 filed on Jun. 9, 2022, the content of which is incorporated by reference in its entirety.

### STATEMENT REGARDING FEDERALLY SPONSORED RESEARCH

This invention was made with government support under AI148511 awarded by the National Institutes of Health. The government has certain rights in the invention.

### REFERENCE TO AN ELECTRONIC SEQUENCE LISTING

The contents of the electronic sequence listing (960296.04414.xml; Size: 100,339 bytes; and Date of Creation: Jun. 20, 2023) is herein incorporated by reference in its entirety.

### BACKGROUND

Recombinant adeno associated virus vectors (rAAV) offer great potential for use as gene therapy vectors for the treatment of monogenic diseases, such as muscular dystrophy and spinal muscular atrophy. rAAV gene therapy vectors offer several advantages over other types of viral vectors due to (1) their ability to persist long-term as a largely unintegrated expression platform, and (2) their inability to elicit significant innate immune responses in the host. However, the widespread use of rAAV vectors in the clinic is limited by our lack of knowledge about how the rAAV genome is chromatinized, where in the host nucleus it persists long-term, and how the vector genome navigates the nuclear milieu. Current gene therapy applications utilize high doses of rAAV vectors (10¹²-10¹³ viral genomes per kg) to ensure proper transgene expression. High doses increase production costs and increase the risk for oncogenic integration and toxicity.

Accordingly, there remains a need in the art for rAAV vectors that drive robust transgene expression in target cells.

### SUMMARY

In a first aspect, the present disclosure provides a construct for producing a recombinant adeno-associated virus (rAAV) vector. The construct comprises: a 5′ inverted terminal repeat (ITR), a first CCCTC-binding factor (CTCF) binding site, a promoter, a transgene, and a 3′ ITR. In embodiments, the construct further comprises a second CTCF binding site. In embodiments, the construct comprises from 5′ to 3′: the 5′ inverted terminal repeat (ITR), the first CCCTC-binding factor (CTCF) binding site, the promoter, the transgene, the second CTCF binding site, and the 3′ ITR. In embodiments, the second CTCF binding site is in the convergent orientation relative to the first CTCF binding site. In embodiments, the CTCF binding site(s) are from a human or a virus.

In embodiments, the virus is selected from the group consisting of: adeno-associated virus (AAV), minute virus of mice (MVM), H1 parvovirus, MmuPV, B19, canine parvovirus, human cytomegalovirus (HCMV)/human herpesvirus 5 strain Merlin, human alphaherpesvirus 1, human herpesvirus 4 type 2 (Epstein-Barr virus type 2), HPV16, herpes simplex virus (HSV), and herpes B virus (HBV).

In embodiments, the CTCF binding site(s) comprise a sequence selected from: SEQ ID NOs:1-28. In embodiments, the first CTCF binding site comprises SEQ ID NO:1 and the second CTCF binding site comprises SEQ ID NO:42. In embodiments, the first and/or second CTCF binding site comprises multiple CTCF binding sequences. In embodiments, the first and/or second CTCF binding site comprises five CTCF binding sequences. In embodiments, the first CTCF binding site comprises SEQ ID NO: 3.

In a second aspect, the present invention provides host cells transduced with a construct described herein.

In a third aspect, the present invention provides rAAV virus particles comprising a construct described herein.

In a fourth aspect, the present invention provides packaging cell lines for producing the virus particles described herein.

In a fifth aspect, the present invention provides a method for producing a modified rAAV virus particle. The method comprises: (a) transducing a host cell with a plasmid comprising a construct described herein, a packaging plasmid, and a helper plasmid; (b) collecting the supernatant and the cells from culture; and (c) isolating virus particles from the supernatant and cells. In embodiments, the method further comprises concentrating the virus particles.

In a sixth aspect, the present invention provides a method of delivering a transgene to a subject in need thereof. The method comprises: administering a modified rAAV virus particle described herein to the subject. In embodiments, the transgene is expressed in a greater proportion of the subject's cells when it is delivered in the modified rAAV vector as compared to when it is delivered in a wild-type rAAV vector. In embodiments, the transgene is expressed at higher levels when it is delivered in the modified rAAV vector as compared to when it is delivered in a wild-type rAAV vector.

### BRIEF DESCRIPTION OF THE DRAWINGS

FIG. 1 is a schematic showing how the modified recombinant adeno-associated virus (rAAV) vectors tested in the Examples were generated. A wild-type rAAV vector comprising a green fluorescent protein (GFP) transgene operably linked to a cytomegalovirus (CMV) promoter was modified via insertion of a first CTCF binding site between the 5′ inverted terminal repeat (ITR) and the CMV promoter and a second CTCF binding site between the GFP transgene and the 3′ ITR.

FIGS. 2A-2B shows the results of a fluorescence-activated cell sorting (FACS) analysis measuring GFP expression in HEK 293 cells transduced with either a (FIG. 2A) wild-type rAAV vector comprising the GFP transgene (WT rAAV) or (FIG. 2B) a modified version of the rAAV vector in which the GFP transgene is flanked by convergent human CTCF binding sites (hCTCF rAAV). These results show that 7.7% of the cells transduced with hCTCF rAAV expressed GFP above background levels, whereas only 3.6% of the cells transduced with WT rAAV expressed GFP above background levels.

FIG. 3 shows the results of a quantitative reverse transcription PCR (RT-qPCR) analysis measuring GFP expression in HEK 293 cells transduced with either WT rAAV or hCTCF rAAV. Mock infected cells (mock) were also analyzed to serve as a negative control. Values were normalized to the levels of housekeeping gene Actb.

FIG. 4 is a schematic depicting the predicted outcomes of inserting CTCF binding sites into rAAV vectors in both the convergent and divergent orientations. When the CTCF binding sites are inserted in the convergent orientation, CTCF binding and dimerization brings together distal DNA elements and results in looping of the intervening sequence. Published data suggests that chromatin loops preferentially form between CTCF binding sites oriented in a convergent manner.

FIGS. 5A-5C is a schematic depicting the difference between an adeno associated virus (AAV) vector (FIG. 5A), a wild-type recombinant adeno-associated virus (rAAV) vector (FIG. 5B), and a modified rAAV vector (FIG. 5C). The triangles represent CTCF binding sites.

FIG. 6 is a schematic of rAAV vectors indicating the locations where the CTCF sites have been inserted (designated as 5′ and 3′; corresponding the Nhel and Xhol restriction enzyme sites). The flags indicate CTCF binding elements and their orientation (convergent or divergent) is shown by their direction.

FIGS. 7A-7E show the FACS analysis of 293T cells transduced for 24 hours with rAAV without insertions (FIG. 7A), and rAAV with CTCF inserts from H1 (FIG. 7B), MVM (FIG. 7C), human (FIG. 7D), and AAV (FIG. 7E). The cells were monitored for levels of GFP positivity. Live cells were first selected by gating on forward and side scatter, which were then assessed for GFP positivity.

FIG. 8 shows the number of GFP transcripts generated per input vector genome. This was computed from rAAV-transduced 293T cells for 24 hours using qRT-PCR. PCR primers were used to determine the ratio of GFP mRNA molecules to that of input vector genomes in the target cells.

### DETAILED DESCRIPTION

The present disclosure provides constructs for producing modified recombinant adeno-associated virus (rAAV) vectors that have improved properties, including increased transgene expression. The constructs comprise one or more CCCTC-binding factor (CTCF) binding sites, which facilitate DNA looping and promote efficient transgene expression. Also provided are modified rAAV virus particles comprising these constructs, methods for producing the modified rAAV virus particles, and methods of using the modified rAAV virus particles to deliver a transgene to a subject.

Recombinant AAV (rAAV) vectors are the platforms of choice for gene therapy to express therapeutic transgenes, and have been designed from Adeno-Associated Viruses (AAVs), that are single-stranded DNA viruses'. Recombinant AAV gene therapy vectors have been designed from AAV parvoviruses by removing all genomic elements, retaining only the Inverted Terminal Repeats (ITRs), which are required to package the transgene in the vector capsid². AAV packaging signals. The resulting rAAV vectors do not contain any of the transcriptional regulatory elements in AAV viruses that regulate AAV gene expression, and as a result do not regulate rAAV expression. This has led to the use of rAAV vectors at high doses in clinical settings.

Expression of genes on the human genome, and from DNA virus genomes, are regulated by packaging of DNA around histones, forming chromatin^{3, 4}. The accessibility of chromatin is regulated by barrier elements, bound by the host protein CTCF. Additionally, CTCF-bound cellular elements loop out intervening DNA molecules, regulating gene expression from promoters by facilitating their 3D interaction with enhancer elements³. The inventors have discovered that the AAV genome is folded into a distinct topological conformation akin to the three-dimensional (3D) structure of the eukaryotic genome, that formation of this 3D structure is required for efficient AAV gene expression, and that formation of the 3D structure is facilitated by binding of the transcription factor CCCTC-binding factor (CTCF) to regulatory elements in the AAV genome. In view of this discovery, the inventors have engineered novel modifications into an rAAV vector that facilitate the formation of 3D structures. Namely, they have introduced one or more binding sites for CTCF into the construct. As demonstrated in the Examples, the modified rAAV vectors drive at least two-fold higher levels of transgene expression in twice as many transduced target cells compared to their wild-type rAAV counterpart, providing surprisingly better transduction results. Thus, the use of the modified rAAV vectors of the present invention improve the use of AAV vectors in gene therapies by reducing the amount of vector that must be administered, which (1) decreases production costs and ultimately increases access to gene therapies, and (2) improves the safety of gene therapies by reducing the chances of oncogenic integration and toxicity.

### Constructs

In a first aspect, the present disclosure provides constructs for producing a modified recombinant adeno-associated virus (rAAV) vector. The constructs comprise: a 5′ inverted terminal repeat (ITR), a first CCCTC-binding factor (CTCF) binding site, a promoter, a transgene, and a 3′ ITR.

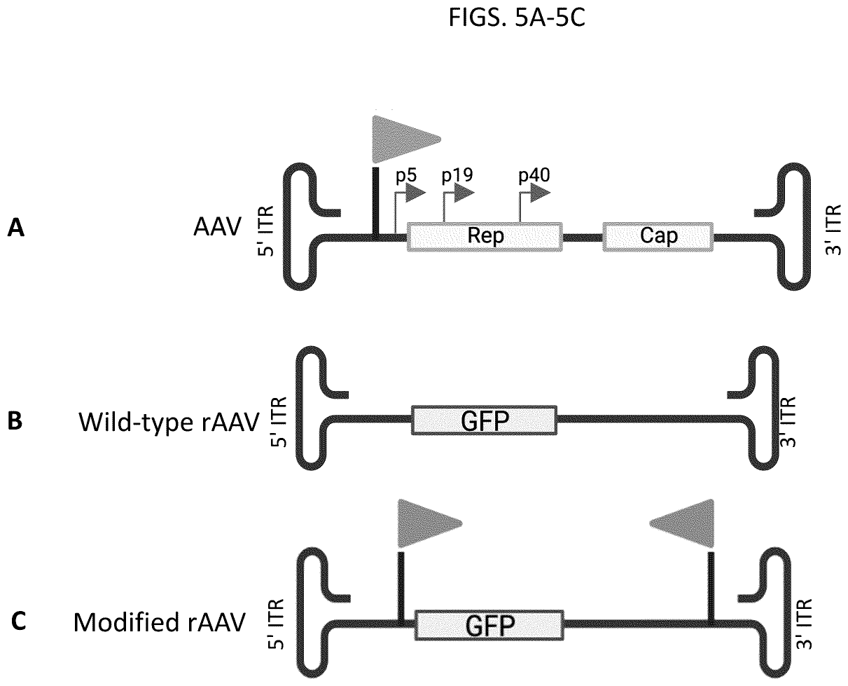

Adeno associated viruses (AAV) are non-pathogenic viruses that belong to the genus Dependoparvovirus. AAV are small, nonenveloped viruses that have a linear single-stranded DNA genome that is approximately 4.7 kilobases (kb) in size. Their genomes encode two distinct sets of proteins: the non-structural replication (Rep) proteins, and the capsid (Cap) proteins that form the structure into which the genome is packaged (FIG. 5A). AAV viruses are replication defective, meaning that the production of AAV virus requires coinfection with helper virus(es). AAV offer several advantages for use as gene therapy vectors: AAV-based gene therapy vectors cause a very mild immune response, can infect both dividing and quiescent cells, and persist in an extrachromosomal state without integrating into the genome of the host cell.

As used herein, a “recombinant adeno-associated virus (rAAV) vector” is an AAV vector in which the Rep/Cap genes and their regulatory sequences have been replaced with a transgene, as depicted in FIG. 5B. As used herein, the term “modified rAAV vector” is used to describe an rAAV vector into which one or more CTCF binding sites has been introduced (FIG. 5C), whereas a “wild-type rAAV vector” is an rAAV vector that lacks CTCF binding sites. Wild-type rAAV vectors genomes persist as linear DNA molecules in the host nucleus, and their expression is regulated solely by the transcriptional regulatory elements (e.g., promoters, enhancers) included in the vector. In contrast, CTCF binding to the CTCF binding sites included in the modified rAAV vectors results in recruitment of transcription factors and/or DNA looping, which can both facilitate more efficient transgene expression. The rAAV vectors of the present invention may comprise a sequence selected from: SEQ ID NOs:29-40, or a sequence having at least 90% identity to any one of SEQ ID NOs:29-40 (Table 4).

As used herein, the term “construct” refers to a recombinant polynucleotide, i.e., a polynucleotide that was formed artificially by combining at least two polynucleotide components from different sources (natural or synthetic). For example, the constructs described herein comprise the coding region of a transgene of interest operably linked to a promoter that (1) is associated with another gene found within the same genome, (2) from the genome of a different species, or (3) is synthetic. Constructs can be generated using conventional recombinant DNA methods. The constructs described herein are single stranded polynucleotides that comprise inverted terminal repeats on their 5′ and 3′ ends.

Constructs may be part of a vector. When referring to a nucleic acid molecule alone, the term “vector” is used herein to describe a nucleic acid molecule capable of transporting another nucleic acid to which it is linked. In contrast, the term “viral vector”, “AAV vector”, or “rAAV vector” is used to describe a virus particle that is used to deliver genetic material (e.g., the constructs of the present invention) into cells.

The constructs of the present invention comprise 5′ and 3′ inverted terminal repeats. “Inverted terminal repeats (ITRs)” are palindromic G-C-rich inverted repeats found on each end of the single stranded AAV genome, which self-base-pair to form unique AAV genome structures. ITRs contain several cis-acting elements that are involved in the initiation of viral DNA replication, as well as binding motifs for cellular transcription factors. Thus, the inclusion of ITRs in the constructs of the present invention allows the constructs to be incorporated into an AAV particle and replicated for viral production.

The constructs of the present invention also comprise one or more CCCTC-binding factor (CTCF) binding sites. CTCF is a transcription factor that regulates the 3D structure of chromatin. CTCF brings specific DNA loci together, forming chromatin loops. Because the 3D structure of DNA influences the regulation of genes, CTCF's activity influences the gene expression. For example, CTCF binding can bridge together promoters and transcription factor-bound enhancers to facilitate transcription initiation. In many cases, two CTCF proteins bound to distinct binding sites dimerize to bring together distal DNA elements. However, in some cases, a single CTCF binding site is sufficient for genome looping. For example, the single CTCF binding site found in the AAV2 genome forms a loop with a region found 2 kb downstream. In these single site instances, CTCF interacts with a different set of architectural proteins, i.e., cohesin and mediator.

As used herein, the term “CTCF binding site” refers to a region of DNA that comprises one or more CTCF binding sequences (i.e., DNA sequences to which CTCF binds). The inventors have generated constructs in which one or more CTCF binding sites (e.g., a first and second CTCF binding site) each comprise five CTCF binding sequences. Thus, in some embodiments, the first and/or second CTCF binding site comprises multiple CTCF binding sequences. For example, the first and/or second CTCF binding site may comprise 2, 3, 4, 5, 6, 7, 8, 9, 10, or more CTCF binding sequences.

The constructs of the present invention also comprise a promoter. As used herein, the term “promoter” refers to a DNA sequence that regulates the transcription of a polynucleotide. Typically, a promoter is a regulatory region that is capable of binding RNA polymerase and initiating transcription of a downstream sequence. However, a promoter may be located at the 5′ or 3′ end, within a coding region, or within an intron of a gene that it regulates. Promoters may be derived in their entirety from a native gene, may be composed of elements derived from multiple regulatory sequences found in nature, or may comprise synthetic DNA segments. It is understood by those skilled in the art that different promoters may direct the expression of a gene in different tissues or cell types, at different stages of development, or in response to different environmental conditions. A promoter is “operably linked” to a polynucleotide if the promoter is connected to the polynucleotide such that it may affect transcription of the polynucleotide.

The constructs of the present invention also comprise a transgene of interest. As used herein, the term “transgene” or “transgene of interest” refers to a gene or genetic material that one wishes to transfer into an organism or a cell thereof. A transgene may encode any protein or functional RNA of interest. Suitable transgenes include those that encode a therapeutic product. For example, the transgene may encode a protein that is lacking due to a genetic disorder or may encode a small interfering RNA (siRNA) that downregulates the expression of a protein that is overexpressed or ectopically expressed due to a genetic disorder. Any suitable transgene for use in gene therapy is contemplated for use in the present disclosure.

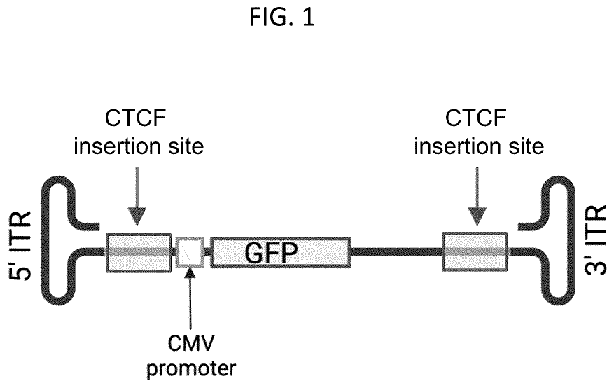

In the Examples, the inventors modified a wild-type rAAV vector comprising a green fluorescent protein (GFP) transgene operably linked to a cytomegalovirus (CMV) promoter by inserting a first CTCF binding site between the 5′ ITR and the CMV promoter and a second CTCF binding site between the GFP transgene and the 3′ ITR as depicted in FIG. 1. Thus, in some embodiments, the constructs further comprise a second CTCF binding site. In specific embodiments, the constructs comprise from 5′ to 3′: the 5′ inverted terminal repeat (ITR), the first CCCTC-binding factor (CTCF) binding site, the promoter, the transgene, the second CTCF binding site, and the 3′ ITR.

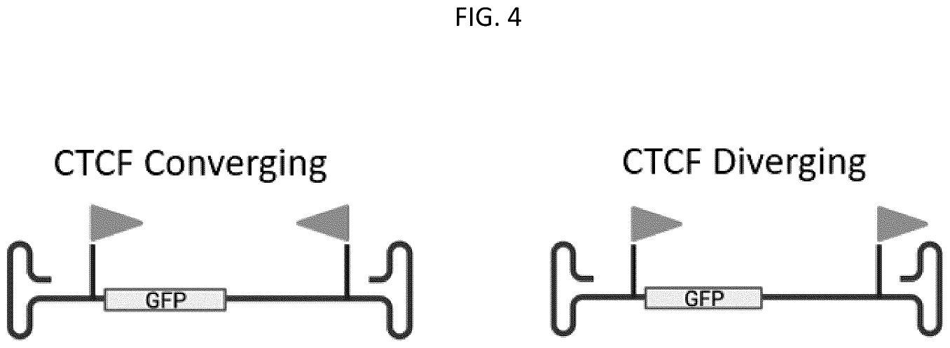

Convergence/divergence of the CTCF binding sites refers to a 5′ to 3′ directionality of CTCF protein binding, and does not refer to the palindromic or non-palindromic nature of the sequences. The inventors have generated constructs in which the two CTCF binding sites are in a convergent orientation as well as constructs in which the two CTCF binding sites are in a divergent orientation. As used herein, the term “convergent orientation” describes two CTCF binding sites that are oriented towards each other, and the term “divergent orientation” describes two CTCF binding sites that are oriented in the same direction or away from each other (see FIG. 4). Published data suggests that chromatin loops preferentially form between CTCF binding sites oriented in a convergent manner. Thus, in some embodiments, the second CTCF binding site is in the convergent orientation relative to the first CTCF binding site.

The CTCF binding sites used in the constructs of the present invention can be from any organism. The inventors have identified a series of suitable CTCF binding sites that are natively found in humans and various viruses. The sequences of these binding sites are provided in Tables 2 and 3. Thus, in some embodiments, the CTCF binding site(s) are from are from a human (e.g., SEQ ID NOs: 1 and 2). In other embodiments, the CTCF binding site(s) are from a virus selected from the group consisting of adeno-associated virus (AAV; e.g., SEQ ID NO: 3), minute virus of mice (MVM; e.g., SEQ ID NOs: 4-6), H1 parvovirus (e.g., SEQ ID NOs: 7-9), mouse papillomavirus (MmuPV) (e.g., SEQ ID NO: 10), B19 (e.g., SEQ ID NO: 11), canine parvovirus (e.g., SEQ ID NO: 12), human cytomegalovirus (HCMV)/human herpesvirus 5 strain Merlin (e.g., SEQ ID NO: 13), human alphaherpesvirus 1 (e.g., SEQ ID NOs: 14-16), human herpesvirus 4 type 2 (Epstein-Barr virus type 2; e.g., SEQ ID NOs: 17-19), human papillomavirus (HPV)16 (e.g., SEQ ID NO: 20), and herpes B virus (HBV) (e.g., SEQ ID NOs: 21 and 22). In some embodiments, the CTCF binding site(s) comprise a sequence selected from: SEQ ID NOs:1-28, or a sequence having at least 90% identity to a sequence selected from: SEQ ID NOs:1-28.

In Example 1, the inventors inserted the human CTCF binding sequence of SEQ ID NO:1 into the 5′ end of the rAAV construct and inserted the CTCF binding sequence of SEQ ID NO:2 into the 3′ end in the convergent orientation. Thus, in some embodiments, the first CTCF binding site comprises SEQ ID NO:1 and the second CTCF binding site comprises SEQ ID NO:2. In one of the constructs of Example 2, the inventors inserted the AAV CTCF binding sequence of SEQ ID NO:3 into the 5′ end of the rAAV construct. Thus, in some embodiments, the construct includes one CTCF binding site of SEQ ID NO:3. In another of the constructs of Example 2, the inventors inserted the human CTCF binding sequence of SEQ ID NO:1 into the 5′ end of the rAAV construct and inserted the human CTCF binding sequence of SEQ ID NO:42 into the 3′ end in the convergent orientation. Thus, in some embodiments, the first CTCF binding site comprises SEQ ID NO:1 and the second CTCF binding site comprises SEQ ID NO:42.

Protein and nucleic acid sequence identities are evaluated using the Basic Local Alignment Search Tool (“BLAST”) which is well known in the art (Karlin and Altschul, 1990, Proc. Natl. Acad. Sci. USA 87: 2267-2268; Altschul et al., 1997, Nucl. Acids Res. 25: 3389-3402). The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. Preferably, the statistical significance of a high-scoring segment pair is evaluated using the statistical significance formula (Karlin and Altschul, 1990), the disclosure of which is incorporated by reference in its entirety. The BLAST programs can be used with the default parameters or with modified parameters provided by the user.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 85% sequence identity to the SEQ ID. Alternatively, percent identity can be any integer from 85% to 100%. More preferred embodiments include at least: 85%, 86%, 87%, 88%, 89%, 90%, 91%, 92%, 93%, 94%, 95%, 96%, 97%, 98% or 99% compared to a reference sequence using the programs described herein; preferably BLAST using standard parameters, as described. These values can be appropriately adjusted to determine corresponding identity of proteins encoded by two nucleotide sequences by taking into account codon degeneracy, amino acid similarity, reading frame positioning, and the like.

“Substantial identity” of amino acid sequences for purposes of this invention normally means polypeptide sequence identity of at least 85%. Preferred percent identity of polypeptides can be any integer from 85% to 100%. More preferred embodiments include at least 85%, 86%, 87%, 88%, 89%, 90%, 91%, 92%, 93%, 94%, 95%, 96%, 97%, 98%, or 99%.

### Host Cells, Virus Particles, and Packaging Cell Lines

In a second aspect, the present invention provides host cells transduced with a construct described herein. As used herein, the term “host cell” refers to any prokaryotic or eukaryotic cell that contains a construct of the present invention. This term also includes cells that have been genetically engineered such that a construct of the present invention is integrated into its genome. The host cell can be a cell line that is used for producing the AAV vectors for use as a gene therapy. Suitable host cells include mammalian cells, including human cells.

The terms “transduced,” “transfected,” and “transformed” all refer to processes by which an exogenous nucleic acid is introduced into a host cell. The term “transduced” specifically refers to the process by which a virus transfers a nucleic acid into a host cell. Plasmids may be used to transfect the construct into a host cell for AAV production along with the helper viruses.

In a third aspect, the present invention provides rAAV virus particles comprising a construct described herein. As used herein, the term “virus particle” refers to a virion consisting of nucleic acid surrounded by a protective protein coat called a capsid.

To generate viral particles, the constructs comprising the rAAV vector are cloned into a plasmid for expression in a host cell. Viral particles may then be generated by helper virus-free co-transfection of HEK 293T cells with three plasmids: (1) an AAV vector comprising a construct of the present invention, (2) a packaging plasmid carrying the AAV Rep and Cap genes, and (3) a helper plasmid carrying the AAV helper functions. For a detailed description of viral production methods, see Ayuso et al. (Gene Ther 17(4):503-10, 2010), which is hereby incorporated by reference in its entirety. Other suitable methods for producing AAV virus particles are well known and understood in the art.

In a fourth aspect, the present invention provides packaging cell lines for producing the virus particles described herein. The term “packaging cell line” is used to refer to a cell line that provides all the proteins necessary for AAV virus production and maturation. Suitable packaging cell lines for use with the present invention include, without limitation, mammalian cells and human cell lines. For example, suitable cell lines include, but are not limited to, HEK 293T cells and HEK 293 cell variants. The packaging cell line should be selected with the method of viral production in mind. For example, cells that have strong adhesion properties should be selected for growth in culture plates, whereas cells lacking adhesion properties should be selected for growth in suspension culture. In some embodiments, the packaging cell line comprises the complement of any genes that have been functionally deleted in the virus particle used to produce the virus, allowing replication incompetent viral particles to be produced.

### Method for Producing a Modified rAAV Virus Particle

In a fifth aspect, the present invention provides methods for producing a modified rAAV virus particle. The methods comprise: (a) transducing a host cell with a plasmid comprising a construct described herein, a packaging plasmid, and a helper plasmid; (b) collecting the supernatant and the cells from culture; and (c) isolating virus particles from the supernatant and cells.

A “plasmid” is a small circular DNA molecule that can replicate independently from chromosomal DNA. In nature, plasmids are commonly found in bacteria, and artificial plasmids are widely used as vectors in molecular cloning.

In the present methods, host cells (e.g., packaging cell lines) are transfected with three plasmids: a plasmid comprising a construct described herein, a packaging plasmid, and a helper plasmid. The term “packaging plasmid” refers to a plasmid that encodes components of the AAV proteins. For rAAV production, the packaging plasmid may encode the AAV genes Rep and Cap. The term “helper plasmid” refers to a plasmid that encodes adenovirus helper functions. Proteins encoded by all three plasmids that are transfected into the host cell in the present methods are required for rAAV production and AAV replication, as is well known in the art.

Virus can be isolated from the supernatant and/or from lysed cells by methods known and understood in the art. Suitable methods for isolating virus from cell culture include, but are not limited to, cesium chloride density gradient centrifugation and affinity purification (e.g., using a porous matrix modified to retain the virus).

In some embodiments, the methods further comprise concentrating the virus. Suitable methods for concentrating virus include, but are not limited to, ultracentrifugation and dialysis.

In some embodiments, the methods further comprise dialyzing the supernatant. For some applications, it may be advantageous to replace the cell culture media present in the supernatant with a solution that is better for long-term storage. Suitable solutions for storage include, but are not limited to, phosphate-buffered saline (PBS), PBS with plutonic acid, saline adjusted to pH 7-7.4 with or without pluronic acid (0.001-0.01%), and Ringer's lactate solution. However, any biocompatible, osmotically balanced, neutral pH fluid should be suitable for storage.

### Method for Delivering a Transgene

In a sixth aspect, the present invention provides methods of delivering a transgene to a subject in need thereof. The methods comprise: administering a modified rAAV virus particle described herein to the subject. By “delivering a transgene” we mean that the methods result in transgene expression in one or more of the subject's cells.

As used herein, the term “administering” refers to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, intradermal administration, intrathecal administration, and subcutaneous administration. Administration can be continuous or intermittent. In some embodiments, the virus particle is administered by vascular injection.

In some embodiments, the virus particle is administered with a pharmaceutically acceptable carrier. “Pharmaceutically acceptable carriers” are known in the art and include, but are not limited to, diluents, preservatives, solubilizers, emulsifiers, liposomes, nanoparticles, and adjuvants. Pharmaceutically acceptable carriers may be aqueous or non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions, and suspensions, including saline and buffered media.

Ideally, the virus particles are administered in a therapeutically effective amount. The term “therapeutically effective amount” refers to an amount sufficient to effect beneficial or desirable biological or clinical results. Methods for determining an effective means of administration and dosage are well known to those of skill in the art and will vary with the formulation used for therapy and the subject (e.g., species, age, health, etc.) being treated. Single or multiple administrations can be carried out with the dose level and pattern being selected by the treating physician. In some embodiments, the virus particle is administered at a dose of 1×10¹² viral genome/kg (vg/kg) or less.

In the Examples, the inventors demonstrate that the introduction of two convergent CTCF binding sites into an rAAV vector comprising a GFP transgene causes the vector to drive GFP expression at higher levels and in a greater proportion of transduced cells. Thus, in some embodiments, the transgene is expressed in a greater proportion of the subject's cells when it is delivered in the modified rAAV vector as compared to when it is delivered in a wild-type rAAV vector. For example, the transgene may be expressed in 1.5 times, 2 times, 3 times, 4 times, or 5 times as many cells as compared to with a wild-type rAAV vector. In some embodiments, the transgene is expressed at higher levels when it is delivered in the modified rAAV vector as compared to when it is delivered in a wild-type rAAV vector. For example, the transgene may be expressed at 1.5 times, 2 times, 3 times, 4 times, or 5 times the level that it is expressed at a wild-type rAAV vector.

Transgene expression can be detected using any suitable method known in the art. For example, when the transgene encodes a protein, the protein product may be detected using an enzyme-linked immunoassay (ELISA), dot blot, western blot, flow cytometry, mass spectrometry, or chromatographic method. When the transgene encodes a functional RNA, the RNA product may be detected using reverse transcription and polymerase chain reaction (RT-PCR) or Northern blotting.

It should be apparent to those skilled in the art that many additional modifications besides those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

As used in this specification and the claims, the singular forms “a,” “an,” and “the” include plural forms unless the context clearly dictates otherwise. Thus, the indefinite articles “a” and “an,” as used herein in the specification and in the claims should be understood to mean “at least one”, unless clearly indicated to the contrary.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term. Where ranges are stated, the endpoints are included within the range unless otherwise stated or otherwise evident from the context.

The phrase “such as” should be interpreted as “for example, including.” Moreover the use of any and all exemplary language, including but not limited to “such as”, is intended merely to better illuminate the invention and does not pose a limitation on the scope of the invention unless otherwise claimed.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.” Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention. No language in the specification should be construed as indicating any non-claimed element as essential to the practice of the invention. It should be understood that descriptions of exemplary embodiments are not intended to limit the invention to the particular forms disclosed, but on the contrary, the intention is to cover all modifications, equivalents and alternatives falling within the spirit and scope of the invention as defined by the appended claims. It should also be understood that, unless clearly indicated to the contrary, in any methods claimed herein that include more than one step or act, the order of the steps or acts of the method is not necessarily limited to the order in which the steps or acts of the method are recited. All methods described herein can be performed in any suitable order unless otherwise indicated herein or otherwise clearly contradicted by context.

The invention will be more fully understood upon consideration of the following non-limiting examples.

### EXAMPLES

### Example 1

In the following Example, the inventors introduce binding sites for the chromatin loop-forming protein CCCTC binding factor (CTCF) into an rAAV vector at sites flanking the rAAV transgene (FIG. 1). They demonstrate that the introduction of these CTCF binding sites enhances transgene expression.

Generation of Modified rAAV Vectors

Human CTCF: The most prevalent CTCF binding site in the human genome was previously identified via chromatin immunoprecipitation sequencing (ChIP-Seq) (Rao et al., Cell, 2015). This study identified pairs of CTCF binding sites that facilitate genome looping, and generated the forward consensus CTCF sequence 5′-CCACNAGGTGGCAG-3′ (SEQ ID NO:24) and the reverse consensus CTCF sequence 5′-CTGCCACCTNGTGG-3′ (SEQ ID NO:25). The inventors cloned CTCF binding sequences into an rAAV plasmid comprising a GFP transgene operably linked to a CMV promoter, which was obtained from Addgene (rAAV-GFP; plasmid #105530). Specifically, a human forward CTCF binding sequence (5′-CCACAAGGTGGCGC-3′; SEQ ID NO:1) was inserted in the 5′ end of the rAAV vector between the 5′ ITR and the CMV promoter, at base pair 205 of the positive-sense strand. A human reverse CTCF binding sequence (5′-CCACCAGGGGGCGG-3′; SEQ ID NO:2) was inserted just downstream of the 3′ ITR, at base pair 2477 of the negative-sense strand, in the convergent orientation. Additionally, a human reverse CTCF binding sequence (5′-GGCGGGGGACCACC-3′; SEQ ID NO:26) was inserted in the divergent orientation at that same location. The sequences of the constructs were confirmed via sequencing (Functional Biosciences).

Viral CTCF: The wild-type AAV2 genome was screened for the presence of CTCF binding sites using the in-silico prediction tool JASPAR (Stormo et al., Quant. Biol, 2013). The inventors discovered that wild-type AAV has a native CTCF binding site (5′-TTGCGACACCATGTGGTCA-3′; SEQ ID NO:3) at the 5′ end of the AAV genome positioned between the 5′ ITR and the p5 promoter (base pairs 166-185) on the positive-sense strand. The inventors detected CTCF binding at this site using ChIP-qPCR. They then inserted this CTCF sequence into the rAAV genome between the 5′ ITR and the CMV promoter, at base pair 205 of the positive-sense strand. They also generated a reverse CTCF binding sequence from this native AAV sequence (i.e., by generating the reverse complement of this sequence) and inserted it in rAAV just upstream of the 3′ ITR, at base pair 2477 of the negative-sense strand. In the convergent orientation, this reverse CTCF sequence is 5′-AACGCTGTGGTACACCAGT-3′ (SEQ ID NO:27) and in the divergent orientation, this sequence is 5′-TGACCACATGGTGTCGCAA-3′ (SEQ ID NO:28).

### Results

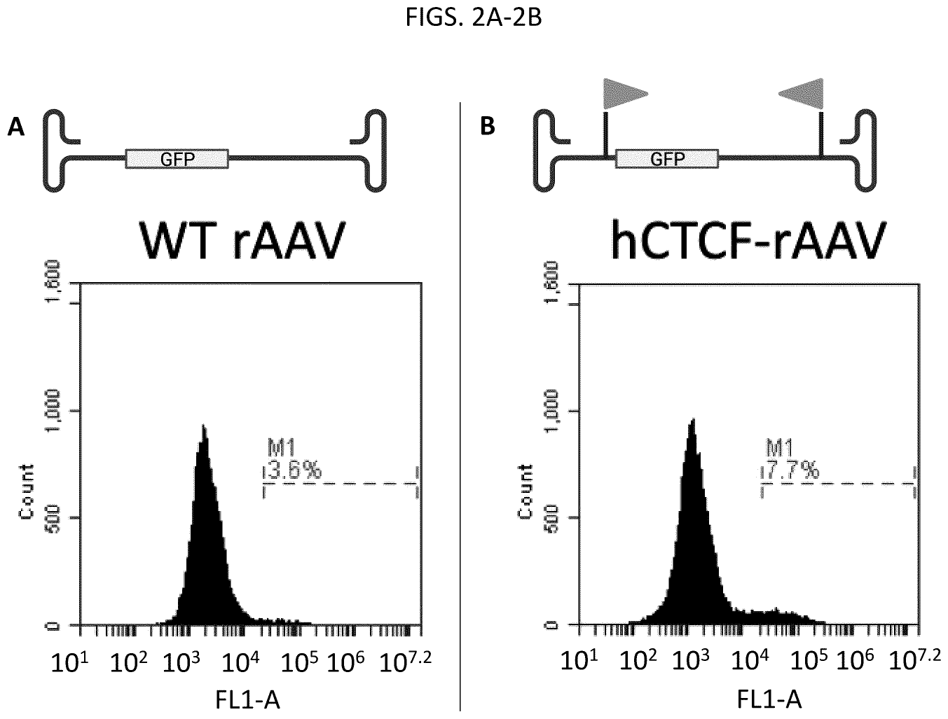

HEK 293 cells were transduced with rAAV vectors comprising a green fluorescent protein (GFP) transgene. The cells were transduced with either a wild-type rAAV vector (comprising no CTCF sequences) or a modified rAAV vector comprising convergent human CTCF binding sequences (Forward: 5′-CCACAAGGTGGCGC-3′ (SEQ ID NO:1); Reverse: 5′-CCACCAGGGGGCGG-3′ (SEQ ID NO:2)) at an MOI of 2,500 viral genomes/cell for 24 hours. A negative control of mock infected cells was used. Cells were collected and resuspended in PBS buffer, and a fluorescence-activated cell sorting (FACS) analysis was performed to measure transgene (i.e., GFP) expression on the cell surface of live cells. The results of this analysis suggest that the addition of the CTCF binding sequences enhances the efficiency transgene expression, as a greater number of the cells transduced with the modified rAAV vector were GFP-positive as compared to the cells transduced with the wild-type rAAV vector (FIG. 2).

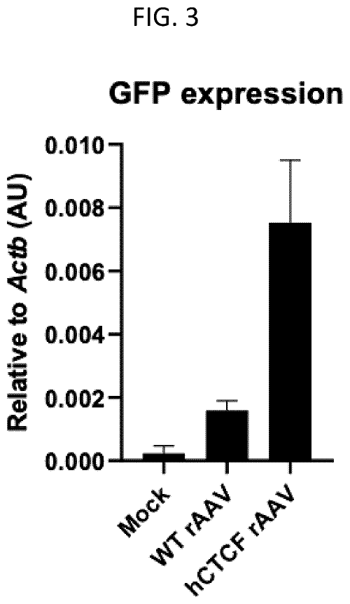

The samples were then subjected to RNA extraction, and qRT-PCR was performed using primers that amplify GFP transcripts to quantify transgene expression. The expression levels were normalized to the levels of the housekeeping gene Actb, and the relative GFP expression levels were compared. The inventors found that GFP expression was significantly enhanced in the cells that were transduced with the modified rAAV vector as compared to the wild-type rAAV vector, suggesting a causal relationship between the CTCF binding sites and increased transgene expression (FIG. 3).

### Example 2

### Additional Viral CTCF Binding Sites

Other DNA viruses and viruses in the parvovirus family have native CTCF binding sites. These CTCF sequences may be able to facilitate looping in rAAVs. For example, minute virus of mice (MVM) is a parvovirus that contains a validated CTCF binding site that is involved in RNA processing and gene expression (Viruses 12(12): 1368, 2020). Many CTCF binding sites have been identified in Kaposi's sarcoma-associated herpesvirus (KSHV) and a single CTCF site has been identified in both Epstein-Barr virus (EBV) and human papillomavirus (HPV). Additionally, the herpes simplex virus type 1 (HSV-1) genome contains CTCF-mediated looping structures that are induced by viral infection.

The inventors have cloned CTCF binding site sequences from other parvovirus genomes (e.g. MVM, AAV subtypes, H1 parvovirus, CPV, and B19) and well-characterized DNA viruses (e.g. HCMV, HSV, EBV, HPV and HBV) into the rAAV vector to determine their impact on transgene expression. The CTCF insertion sequences used in the vectors are listed in Table 1.

### Identification of Cis-Elements

The inventors have scanned the genomes of DNA viruses, including parvoviruses such as AAV, MVM, H1, B19, CPV, as well as herpesviruses such as EBV, HSV, HCMV and tumor viruses such as HPV16 and HBV, to identify CTCF binding sites in-silico using the JASPAR online database of transcription factor binding sites⁵. These online screens identified the viral CTCF binding elements in DNA viruses. The inventors additionally identified published CTCF sites on the human genome that have been previously identified using CTCF ChIP-seq genome-wide³.

Generation of rAAV Vectors Containing Chromatin Modifying Cis-Elements

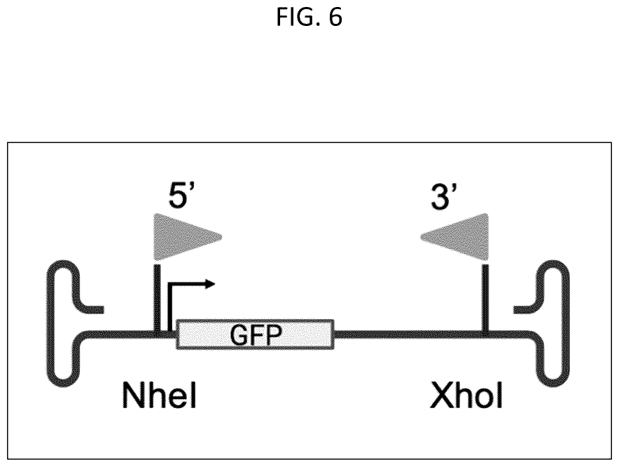

The inventors cloned the identified CTCF binding elements into the 5′ end of the rAAV vector expressing a GFP transgene from a CMV promoter as shown in FIG. 6 (labelled as 5′ insert into the NheI restriction enzyme site). They additionally cloned these CTCF sequences into the 3′ CTCF insert site, downstream of the poly-A tail (labelled as 3′ CTCF insert into the XhoI restriction enzyme site). These sequence orientations were varied according to their forward version (labelled as F in Table 1) and in the reverse orientation (labelled as R in Table 1). A subset of the sequence inserts contained multiple CTCF binding elements (designated by multiple F's and R's in Table 1). Convergent CTCF orientations in Table 1 are labelled as “con” and non-convergent CTCF orientations are designated as “noncon”.

TABLE 1
Location and sequences of rAAV gene therapy
containingvector constructs TCF insertions
that have been successfully generated

| | | | 3′ CTCF |
|---|---|---|---|
| | | 5′ CTCF insert | insert sequence |
| | | sequence (position | (position |
| | CTCF | 200 of SEQ ID | 2472 of SEQ ID |
| Vector | site | NO: 29, on the | NO: 29, on the |
| number | origin | NheI site) | XhoI site) |
| | AAV | | |
| | CTCF | | |
| 1 | 5F_C2 | TTGCGACACCATGTGGTCA | |
| | | (SEQ ID NO: 3) | |
| 2 | 5F_3R | TTGCGACACCATGTGGTCA | ACTGGTGTACCACAG |
| | con | (SEQ ID NO: 3) | CGTT |
| | | | (SEQ ID NO: 41) |
| 3 | 5F_3F | TTGCGACACCATGTGGTCA | TTGCGACACCATGTG |
| | noncon | (SEQ ID NO: 3) | GTCA |
| | | | (SEQ ID NO: 3) |
| | hCTCF | | |
| 4 | 5F_C1 | CCACAAGGTGGCGC | |
| | | (SEQ ID NO: 1) | |
| 5 | 5F_3R | CCACAAGGTGGCGC | CCGCCCCCTGGTGG |
| | con | (SEQ ID NO: 1) | (SEQ ID NO: 42) |
| | | | (reverse |
| | | | complement |
| | | | of SEQ |
| | | | ID NO: 2) |
| 6 | 5F_3F | CCACAAGGTGGCGC | GGTGGTCCCCCGCC |
| | noncon | (SEQ ID NO: 1) | (SEQ ID NO: 43) |
| | MVM#4 | | |
| 7 | 5F | TTGCTCACTAGATGGCGCT | |
| | | C (SEQ ID NO: 44) | |
| 8 | 5R | CTCGCGGTAGATCACTCGT | |
| | | T (SEQ ID NO: 4) | |
| | MVM#5 | | |
| 9 | 5F | CCACCACTAAATGGCATTC | |
| | | TT (SEQ ID NO: 5) | |
| 10 | 5R | TTCTTACGGTAAATCACCA | |
| | | CC (SEQ ID NO: 45) | |
| 11 | 5F_3F | CCACCACTAAATGGCATTC | TTCTTACGGTAAATC |
| | | TT (SEQ ID NO: 5) | ACCACC |
| | | | (SEQ ID NO: 46) |
| 12 | 5F_3R | CCACCACTAAATGGCATTC | CCACCACTAAATGGC |
| | | TT (SEQ ID NO: 5) | ATTCTT |
| | | | (SEQ ID NO: 5) |
| | H1 | | |
| | Parvo | | |
| 13 | 5F | AGTCCACCAAGGGACGGAG | |
| | | (SEQ ID NO: 9) | |
| 14 | 5F_3F | AGTCCACCAAGGGACGGAG | GAGGCAGGGAACCAC |
| | | (SEQ ID NO: 9) | CTGA |
| | | | (SEQ ID NO: 47) |
| 15 | 5F_3R | AGTCCACCAAGGGACGGAG | AGTCCACCAAGGGAC |
| | | (SEQ ID NO: 9) | GGAG |
| | | | (SEQ ID NO: 9) |
| 16 | 5F_3RF | AGTCCACCAAGGGACGGAG | AGTCCACCAAGGGAC |
| | | (SEQ ID NO: 9) | GGAGGAGGCAGGGAA |
| | | | CCACCTGA |
| | | | (SEQ ID NO: 48) |
| 17 | 5R | GAGGCAGGGAACCACCTGA | |
| | | (SEQ ID NO: 49) | |
| | HBV | | |
| | CTCF 1 | | |
| 18 | 5RR | ACCTAGGTTGACCACCAGC | |
| | | ACCTAGGTTGACCACCAGC | |
| | | (SEQ ID NO: 50) | |
| 19 | 5RRR | ACCTAGGTTGACCACCAGC | |
| | | ACCTAGGTTGACCACCAGC | |
| | | ACCTAGGTTGACCACCAGC | |
| | | (SEQ ID NO: 51) | |
| | HBV | | |
| | CTCF 2 | | |
| 20 | 5F | TCTACAGCATGGGGCAGAA | |
| | | (SEQ ID NO: 22) | |
| | HBV | | |
| | CTCF 1 | | |
| 21 | 5RF | ACCTAGGTTGACCACCAGC | |
| | | CGACCACCAGTTGGATCCA | |
| | | (SEQ ID NO: 52) | |
| 22 | 5RFFR | ACCTAGGTTGACCACCAGC | |
| | | CGACCACCAGTTGGATCCA | |
| | | CGACCACCAGTTGGATCCA | |
| | | CGACCACCAGTTGGATCCA | |
| | | (SEQ ID NO: 53) | |
| | EBV | | |
| | CTCF2 | | |
| 23 | 5FFFFF | CACCCAACAGGTGGTGAAA | |
| | | CACCCAACAGGTGGTGAAA | |
| | | CACCCAACAGGTGGTGAAA | |
| | | CACCCAACAGGTGGTGAAA | |
| | | CACCCAACAGGTGGTGAAA | |
| | | (SEQ ID NO: 54) | |
| 24 | 5RRRRR | AAAGTGGTGGACAACCCAC | |
| | R | AAAGTGGTGGACAACCCAC | |
| | | AAAGTGGTGGACAACCCAC | |
| | | AAAGTGGTGGACAACCCAC | |
| | | AAAGTGGTGGACAACCCAC | |
| | | AAAGTGGTGGACAACCCAC | |
| | | (SEQ ID NO: 55) | |
| | EBV | | |
| | CTCF3 | | |
| 25 | 5R | ACCGTGGTGTACCACGGTT | |
| | | (SEQ ID NO: 56) | |
| 26 | 5FF | TTGGCACCATGTGGTGCCA | |
| | | TTGGCACCATGTGGTGCCA | |
| | | (SEQ ID NO: 57) | |
| | CPV | | |
| | CTCF | | |
| 27 | 5F | CAACCAGGAGGTGAAAATC | |
| | | (SEQ ID NO: 12) | |
| 28 | 5R | CTAAAAGTGGAGGACCAAC | |
| | | (SEQ ID NO: 58) | |
| 29 | 5FF | CAACCAGGAGGTGAAAATC | |
| | | CAACCAGGAGGTGAAAATC | |
| | | (SEQ ID NO: 59) | |
| | HSV | | |
| | CTCF2 | | |
| 30 | 5F | CCACCGGCGGGGGGCGGCG | |
| | | (SEQ ID NO: 15) | |
| 31 | 5FF | CCACCGGCGGGGGGGGCG | |
| | | CCACCGGCGGGGGGCGGCG | |
| | | (SEQ ID NO: 60) | |
| 32 | 5FFF | CCACCGGCGGGGGGCGGCG | |
| | | CCACCGGCGGGGGGCGGCG | |
| | | CCACCGGCGGGGGGCGGCG | |
| | | (SEQ ID NO: 61) | |
| | B19 | | |
| 33 | 5F | ATACTGGGGGATAACCACC | |
| | | (SEQ ID NO: 62) | |
| 34 | 5FF | ATACTGGGGGATAACCACC | |
| | | ATACTGGGGGATAACCACC | |
| | | (SEQ ID NO: 63) | |
| | HCMV | | |
| 35 | 5F | CGACCCGCACATGGCGCTG | |
| | | (SEQ ID NO: 13) | |
| | HPV16 | | |
| 36 | 5F | TAACCACCAGGTGGTGCCA | |
| | | (SEQ ID NO: 20) | |

| | | |
|---|---|---|
| 37 | 5RR | ACCGTGGTGGACCACCAAT |
| | | ACCGTGGTGGACCACCAAT |
| | | (SEQ ID NO: 64) |

### Vector Production

rAAV vectors were produced in HEK 293T cells by cotransfecting them with Rep/Cap plasmids (expressing AAV Rep and Cap proteins) and pHelper plasmids (expressing essential Adenovirus proteins such as E1, E2, E4ORF6 and VA-RNA) for 6-7 days. Vectors were harvested from the producer cells by rapid freeze/thaw cycles, DNAse treated and transduced into target 293T cells⁶. These cells were assessed for GFP expression by FACS and qRT-PCR as described below.

### Assessing the Number of Cells That are GFP Positive

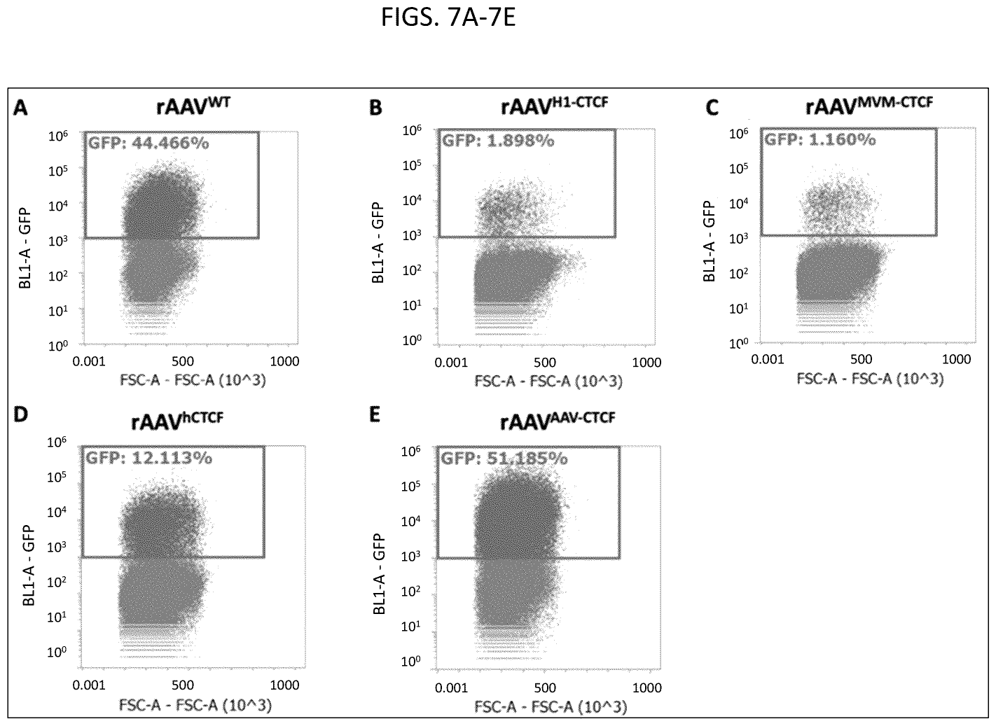

rAAV-GFP vectors were used to transduce HEK 293T cells for 24 hours. They were subsequently monitored for GFP positivity using FACS analysis after gating on the live cells by forward scatter and side scatter. As shown in FIG. 7A, 44.4% of cells transduced with the wild-type rAAV vector without insertions were GFP positive at 24 hpi. Cells transduced with vectors containing the CTCF inserts from H1 parvovirus and MVM parvovirus were respectively 1.8% and 1.1% GFP positive (FIG. 7B; corresponding to Vector number 13 in Table 1 and FIG. 7C; corresponding to Vector number 7 in Table 1). Cells transduced with human CTCF binding elements in a convergent orientation were 12% GFP positive (FIG. 7D, corresponding to Vector number 5 in Tables 1 and 4 (SEQ ID NO: 37)). Strikingly, cells transduced with an rAAV vector containing the AAV CTCF binding element were 51% GFP positive (FIG. 7E, corresponding to Vector number 1 in Tables 1 and 4 (SEQ ID NO: 29)). Notably, the H1, MVM and AAV CTCF insertions were in the 5′ end of the genome only, whereas the hCTCF insertion was at both ends of the genome in a convergent orientation. These findings suggested that CTCF binding site insertions modulate the transduction efficiency of gene therapy vectors in the host.

### Vector Expression Efficiency per Vector Genome

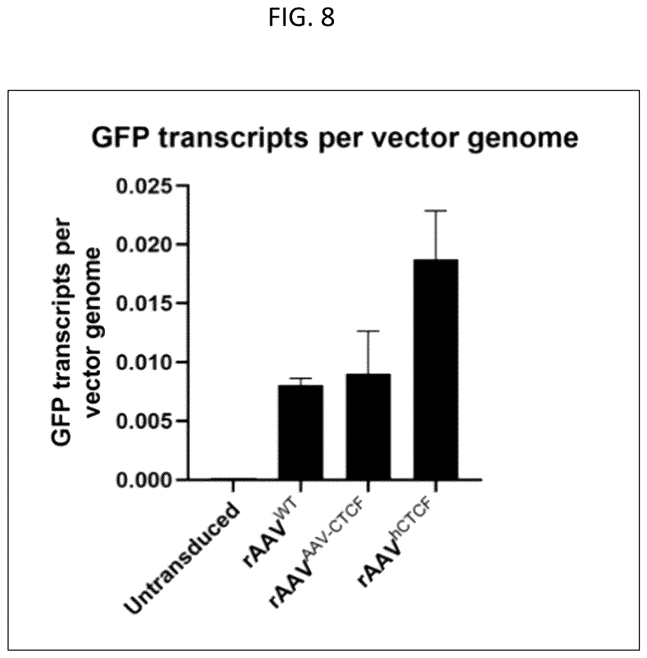

To monitor the ability of novel rAAV gene therapy vectors to express in transduced cells, the inventors normalized the GFP transcript levels generated in target cells to that of input vector genomes. They compared the mRNA molecules per input vector in the current iteration of rAAV vectors to that of the novel constructs, focusing on the constructs containing the convergent hCTCF sites (Vector number 5 in Table 1) and the AAV CTCF sites (Vector number 1 in Table 1). Compared with the current rAAV vectors, rAAV^{AAV-CTCF} yielded similar levels of GFP mRNA per vector whereas rAAV^{hCTCF} vectors expressed at double these levels (FIG. 8). These findings indicate that the CTCF binding elements other than those derived from AAV in rAAV vectors are able to increase the expression capacity of rAAV genomes in individual cells as well as increase the number of cells capable of expressing the rAAV genome.

Examples of other viral CTCF binding sequences include those listed in Tables 2 and 3. Sequences of rAAV vectors are provided in Table 4.

TABLE 2
Native CTCF binding site sequences found in
human and viral genomes

| | | | Location | Strand |
|---|---|---|---|---|
| SEQ ID | CTCF sequence | | in | (Fwd/ |
| NO: | (5′ to 3′) | Organism | genome | Rev) |
| 1 | CCACAAGGTGGCGC | Human | chr1 | Fwd |
| 2 | CCACCAGGGGGCGG | Human | chr1 | Rev |
| 3 | TTGCGACACCATGTGGTCA | AAV | 166 | Fwd |
| 4 | CTCGCGGTAGATCACTCGT | Minute virus of mice | 990 | Rev |
| | T | (MVM) | | |
| 5 | CCACCACTAAATGGCATTC | Minute virus of mice | 4460 | Fwd |
| | TT | (MVM) | | |
| 6 | CTCGCGGTAGATCACTCGT | Minute virus of mice | 990 | Rev |
| | T | (MVM) | | |
| 7 | TGCTCACTAGATGGCGCTC | H1 parvovirus | 900 | Rev |
| 8 | TGCACAGCAGAGGACTCTG | H1 parvovirus | 2682 | Fwd |
| 9 | AGTCCACCAAGGGACGGAG | H1 parvovirus | 828 | Fwd |
| 10 | TGAACAGTAGGAGTCAGTT | MmuPV | 2966 | Fwd |
| 11 | CCACCAATAGGGGGTCATA | B19 | 1516 | Rev |
| 12 | CAACCAGGAGGTGAAAATC | Canine parvovirus | 1220 | Fwd |
| 13 | CGACCCGCACATGGCGCTG | HCMV; Human | 2205 | Fwd |
| | | herpesvirus 5 strain | | |
| | | Merlin | | |
| 14 | AGACCACCAGGTGGCGCAC | Human | 1068 | Rev |
| | | alphaherpesvirus 1 | | |
| 15 | CCACCGGCGGGGGGCGGCG | Human | 655 | Rev |
| | | alphaherpesvirus 1 | | |
| 16 | CGGGCGGCCGGGGGGGGCG | Human | 543 | Rev |
| | | alphaherpesvirus 1 | | |
| 17 | TGGCCAAAAGACGGCGGTT | Human herpesvirus 4 | 2226 | Fwd |
| | | type 2 (Epstein-Barr | | |
| | | virus type 2) | | |
| 18 | CACCCAACAGGTGGTGAAA | Human herpesvirus 4 | 2020 | Fwd |
| | | type 2 (Epstein-Barr | | |
| | | virus type 2) | | |
| 19 | TTGGCACCATGTGGTGCCA | Human herpesvirus 4 | 407 | Fwd |
| | | type 2 (Epstein-Barr | | |
| | | virus type 2) | | |
| 20 | TAACCACCAGGTGGTGCCA | HPV16 | 2053 | Fwd |
| 21 | CGACCACCAGTTGGATCCA | HBV | 1079 | Fwd |
| 22 | TCTACAGCATGGGGCAGAA | HBV | 1027 | Fwd |

TABLE 3
Other CTCF binding site sequences used in the Examples

| SEQ ID | CTCF sequence | |
|---|---|---|
| NO: | (5′ to 3′) | Description |
| 24 | CCACNAGGTGGCAG | Human forward CTCF consensus sequence |
| 25 | CTGCCACCTNGTGG | Human reverse CTCF consensus sequence |
| 26 | GGCGGGGGACCACC | Human reverse CTCF sequence in the |
| | | divergent orientation (i.e., SEQ ID |
| | | NO: 2 in reverse order) |
| 27 | AACGCTGTGGTACA | AAV Reverse CTCF sequence, generated |
| | CCAGT | for use the native AAV CTCF sequence, |
| | | convergent orientation |
| 28 | TGACCACATGGTGT | AAV Reverse CTCF sequence, generated |
| | CGCAA | for use the native AAV CTCF sequence, |
| | | divergent orientation (i.e., SEQ ID |
| | | NO: 27 in reverse order) |

TABLE 4
rAAV sequences. The inserted CTCF binding
sites are underlined.

| SEQ ID NO: | Description and Sequence |
|---|---|
| 29 | rAAV with forward AAV CTCF sequence knocked in |
| | on positive-sense strand (Vector 1, Table 1) |
| | CCGCTCGCTCGCTCACTGAGGCCGCCCGGGCAAAGCCCGGGCGTCGGGC |
| | GACCTTTGGTCGCCCGGCCTCAGTGAGCGAGCGAGCGCGCAGAGAGGGA |
| | GTGGCCAACTCCATCACTAGGGGTTCCTTGTAGTTAATGATTAACCCGC |
| | CATGCTACTTATCTACGTAGCCATGCTCTAGGAAGATCGGAATTCGCCC |
| | TTAAGCTAGTTGCGACACCATGTGGTCACTAGCTAGTTATTAATAGTAA |
| | TCAATTACGGGGTCATTAGTTCATAGCCCATATATGGAGTTCCGCGTTA |
| | CATAACTTACGGTAAATGGCCCGCCTGGCTGACCGCCCAACGACCCCCG |
| | CCCATTGACGTCAATAATGACGTATGTTCCCATAGTAACGCCAATAGGG |
| | ACTTTCCATTGACGTCAATGGGTGGAGTATTTACGGTAAACTGCCCACT |
| | TGGCAGTACATCAAGTGTATCATATGCCAAGTACGCCCCCTATTGACGT |
| | CAATGACGGTAAATGGCCCGCCTGGCATTATGCCCAGTACATGACCTTA |
| | TGGGACTTTCCTACTTGGCAGTACATCTACGTATTAGTCATCGCTATTA |
| | CCATGGTGATGCGGTTTTGGCAGTACATCAATGGGCGTGGATAGCGGTT |
| | TGACTCACGGGGATTTCCAAGTCTCCACCCCATTGACGTCAATGGGAGT |
| | TTGTTTTGGCACCAAAATCAACGGGACTTTCCAAAATGTCGTAACAACT |
| | CCGCCCCATTGACGCAAATGGGCGGTAGGCGTGTACGGTGGGAGGTCTA |
| | TATAAGCAGAGCTGGTTTAGTGAACCGTCAGATCCTGCAGAAGTTGGTC |
| | GTGAGGCACTGGGCAGGTAAGTATCAAGGTTACAAGACAGGTTTAAGGA |
| | GACCAATAGAAACTGGGCTTGTCGAGACAGAGAAGACTCTTGCGTTTCT |
| | GATAGGCACCTATTGGTCTTACTGACATCCACTTTGCCTTTCTCTCCAC |
| | AGGTGTCCAGGCGGCCGCCATGGTGAGCAAGGGCGAGGAGCTGTTCACC |
| | GGGGTGGTGCCCATCCTGGTCGAGCTGGACGGCGACGTAAACGGCCACA |
| | AGTTCAGCGTGTCCGGCGAGGGCGAGGGCGATGCCACCTACGGCAAGCT |
| | GACCCTGAAGTTCATCTGCACCACCGGCAAGCTGCCCGTGCCCTGGCCC |
| | ACCCTCGTGACCACCCTGACCTACGGCGTGCAGTGCTTCAGCCGCTACC |
| | CCGACCACATGAAGCAGCACGACTTCTTCAAGTCCGCCATGCCCGAAGG |
| | CTACGTCCAGGAGCGCACCATCTTCTTCAAGGACGACGGCAACTACAAG |
| | ACCCGCGCCGAGGTGAAGTTCGAGGGCGACACCCTGGTGAACCGCATCG |
| | AGCTGAAGGGCATCGACTTCAAGGAGGACGGCAACATCCTGGGGCACAA |
| | GCTGGAGTACAACTACAACAGCCACAACGTCTATATCATGGCCGACAAG |
| | CAGAAGAACGGCATCAAGGTGAACTTCAAGATCCGCCACAACATCGAGG |
| | ACGGCAGCGTGCAGCTCGCCGACCACTACCAGCAGAACACCCCCATCGG |
| | CGACGGCCCCGTGCTGCTGCCCGACAACCACTACCTGAGCACCCAGTCC |
| | GCCCTGAGCAAAGACCCCAACGAGAAGCGCGATCACATGGTCCTGCTGG |
| | AGTTCGTGACCGCCGCCGGGATCACTCTCGGCATGGACGAGCTGTACAA |
| | GTAATAAGCTTGGATCCAATCAACCTCTGGATTACAAAATTTGTGAAAG |
| | ATTGACTGGTATTCTTAACTATGTTGCTCCTTTTACGCTATGTGGATAC |
| | GCTGCTTTAATGCCTTTGTATCATGCTATTGCTTCCCGTATGGCTTTCA |
| | TTTTCTCCTCCTTGTATAAATCCTGGTTGCTGTCTCTTTATGAGGAGTT |
| | GTGGCCCGTTGTCAGGCAACGTGGCGTGGTGTGCACTGTGTTTGCTGAC |
| | GCAACCCCCACTGGTTGGGGCATTGCCACCACCTGTCAGCTCCTTTCCG |
| | GGACTTTCGCTTTCCCCCTCCCTATTGCCACGGCGGAACTCATCGCCGC |
| | CTGCCTTGCCCGCTGCTGGACAGGGGCTCGGCTGTTGGGCACTGACAAT |
| | TCCGTGGTGTTGTCGGGGAAATCATCGTCCTTTCCTTGGCTGCTCGCCT |
| | GTGTTGCCACCTGGATTCTGCGCGGGACGTCCTTCTGCTACGTCCCTTC |
| | GGCCCTCAATCCAGCGGACCTTCCTTCCCGCGGCCTGCTGCCGGCTCTG |
| | CGGCCTCTTCCGCGTCTTCGAGATCTGCCTCGACTGTGCCTTCTAGTTG |
| | CCAGCCATCTGTTGTTTGCCCCTCCCCCGTGCCTTCCTTGACCCTGGAA |
| | GGTGCCACTCCCACTGTCCTTTCCTAATAAAATGAGGAAATTGCATCGC |
| | ATTGTCTGAGTAGGTGTCATTCTATTCTGGGGGGTGGGGTGGGGCAGGA |
| | CAGCAAGGGGGAGGATTGGGAAGACAATAGCAGGCATGCTGGGGACTCG |
| | ATCGAGTTAAGGGCGAATTCCCGATAAGGATCTTCCTAGAGCATGGCTA |
| | CGTAGATAAGTAGCATGGCGGGTTAATCATTAACTACAAGGAACCCCTA |
| | GTGATGGAGTTGGCCACTCCCTCTCTGCGCGCTCGCTCGCTCACTGAGG |
| | CCGGGCGACCAAAGGTCGCCCGACGCCCGGGCTTTGCCCGGGCGGCCTC |
| | AGTGAGCGAGCGAGCGCGCAG |
| 30 | rAAV with reverse AAV CTCF sequence knocked |
| | in on positive-sense strand |
| | CCGCTCGCTCGCTCACTGAGGCCGCCCGGGCAAAGCCCGGGCGTCGGGC |
| | GACCTTTGGTCGCCCGGCCTCAGTGAGCGAGCGAGCGCGCAGAGAGGGA |
| | GTGGCCAACTCCATCACTAGGGGTTCCTTGTAGTTAATGATTAACCCGC |
| | CATGCTACTTATCTACGTAGCCATGCTCTAGGAAGATCGGAATTCGCCC |
| | TTAAGCTAGCTAGCTAGTTATTAATAGTAATCAATTACGGGGTCATTAG |
| | TTCATAGCCCATATATGGAGTTCCGCGTTACATAACTTACGGTAAATGG |
| | CCCGCCTGGCTGACCGCCCAACGACCCCCGCCCATTGACGTCAATAATG |
| | ACGTATGTTCCCATAGTAACGCCAATAGGGACTTTCCATTGACGTCAAT |
| | GGGTGGAGTATTTACGGTAAACTGCCCACTTGGCAGTACATCAAGTGTA |
| | TCATATGCCAAGTACGCCCCCTATTGACGTCAATGACGGTAAATGGCCC |
| | GCCTGGCATTATGCCCAGTACATGACCTTATGGGACTTTCCTACTTGGC |
| | AGTACATCTACGTATTAGTCATCGCTATTACCATGGTGATGCGGTTTTG |
| | GCAGTACATCAATGGGCGTGGATAGCGGTTTGACTCACGGGGATTTCCA |
| | AGTCTCCACCCCATTGACGTCAATGGGAGTTTGTTTTGGCACCAAAATC |
| | AACGGGACTTTCCAAAATGTCGTAACAACTCCGCCCCATTGACGCAAAT |
| | GGGCGGTAGGCGTGTACGGTGGGAGGTCTATATAAGCAGAGCTGGTTTA |
| | GTGAACCGTCAGATCCTGCAGAAGTTGGTCGTGAGGCACTGGGCAGGTA |
| | AGTATCAAGGTTACAAGACAGGTTTAAGGAGACCAATAGAAACTGGGCT |
| | TGTCGAGACAGAGAAGACTCTTGCGTTTCTGATAGGCACCTATTGGTCT |
| | TACTGACATCCACTTTGCCTTTCTCTCCACAGGTGTCCAGGCGGCCGCC |
| | ATGGTGAGCAAGGGCGAGGAGCTGTTCACCGGGGTGGTGCCCATCCTGG |
| | TCGAGCTGGACGGCGACGTAAACGGCCACAAGTTCAGCGTGTCCGGCGA |
| | GGGCGAGGGCGATGCCACCTACGGCAAGCTGACCCTGAAGTTCATCTGC |
| | ACCACCGGCAAGCTGCCCGTGCCCTGGCCCACCCTCGTGACCACCCTGA |
| | CCTACGGCGTGCAGTGCTTCAGCCGCTACCCCGACCACATGAAGCAGCA |
| | CGACTTCTTCAAGTCCGCCATGCCCGAAGGCTACGTCCAGGAGCGCACC |
| | ATCTTCTTCAAGGACGACGGCAACTACAAGACCCGCGCCGAGGTGAAGT |
| | TCGAGGGCGACACCCTGGTGAACCGCATCGAGCTGAAGGGCATCGACTT |
| | CAAGGAGGACGGCAACATCCTGGGGCACAAGCTGGAGTACAACTACAAC |
| | AGCCACAACGTCTATATCATGGCCGACAAGCAGAAGAACGGCATCAAGG |
| | TGAACTTCAAGATCCGCCACAACATCGAGGACGGCAGCGTGCAGCTCGC |
| | CGACCACTACCAGCAGAACACCCCCATCGGCGACGGCCCCGTGCTGCTG |
| | CCCGACAACCACTACCTGAGCACCCAGTCCGCCCTGAGCAAAGACCCCA |
| | ACGAGAAGCGCGATCACATGGTCCTGCTGGAGTTCGTGACCGCCGCCGG |
| | GATCACTCTCGGCATGGACGAGCTGTACAAGTAATAAGCTTGGATCCAA |
| | TCAACCTCTGGATTACAAAATTTGTGAAAGATTGACTGGTATTCTTAAC |
| | TATGTTGCTCCTTTTACGCTATGTGGATACGCTGCTTTAATGCCTTTGT |
| | ATCATGCTATTGCTTCCCGTATGGCTTTCATTTTCTCCTCCTTGTATAA |
| | ATCCTGGTTGCTGTCTCTTTATGAGGAGTTGTGGCCCGTTGTCAGGCAA |
| | CGTGGCGTGGTGTGCACTGTGTTTGCTGACGCAACCCCCACTGGTTGGG |
| | GCATTGCCACCACCTGTCAGCTCCTTTCCGGGACTTTCGCTTTCCCCCT |
| | CCCTATTGCCACGGCGGAACTCATCGCCGCCTGCCTTGCCCGCTGCTGG |
| | ACAGGGGCTCGGCTGTTGGGCACTGACAATTCCGTGGTGTTGTCGGGGA |
| | AATCATCGTCCTTTCCTTGGCTGCTCGCCTGTGTTGCCACCTGGATTCT |
| | GCGCGGGACGTCCTTCTGCTACGTCCCTTCGGCCCTCAATCCAGCGGAC |
| | CTTCCTTCCCGCGGCCTGCTGCCGGCTCTGCGGCCTCTTCCGCGTCTTC |
| | GAGATCTGCCTCGACTGTGCCTTCTAGTTGCCAGCCATCTGTTGTTTGC |
| | CCCTCCCCCGTGCCTTCCTTGACCCTGGAAGGTGCCACTCCCACTGTCC |
| | TTTCCTAATAAAATGAGGAAATTGCATCGCATTGTCTGAGTAGGTGTCA |
| | TTCTATTCTGGGGGGTGGGGTGGGGCAGGACAGCAAGGGGGAGGATTGG |
| | GAAGACAATAGCAGGCATGCTGGGGACTCGAACTGGTGTACCACAGCGT |
| | TTCGAGTTAAGGGCGAATTCCCGATAAGGATCTTCCTAGAGCATGGCTA |
| | CGTAGATAAGTAGCATGGCGGGTTAATCATTAACTACAAGGAACCCCTA |
| | GTGATGGAGTTGGCCACTCCCTCTCTGCGCGCTCGCTCGCTCACTGAGG |
| | CCGGGCGACCAAAGGTCGCCCGACGCCCGGGCTTTGCCCGGGCGGCCTC |
| | AGTGAGCGAGCGAGCGCGCAG |
| 31 | rAAV with AAV CTCF convergent sequence |
| | knocked in on positive-sense strand |
| | CCGCTCGCTCGCTCACTGAGGCCGCCCGGGCAAAGCCCGGGCGTCGGGC |
| | GACCTTTGGTCGCCCGGCCTCAGTGAGCGAGCGAGCGCGCAGAGAGGGA |
| | GTGGCCAACTCCATCACTAGGGGTTCCTTGTAGTTAATGATTAACCCGC |
| | CATGCTACTTATCTACGTAGCCATGCTCTAGGAAGATCGGAATTCGCCC |
| | TTAAGCTAGTTGCGACACCATGTGGTCACTAGCTAGTTATTAATAGTAA |
| | TCAATTACGGGGTCATTAGTTCATAGCCCATATATGGAGTTCCGCGTTA |
| | CATAACTTACGGTAAATGGCCCGCCTGGCTGACCGCCCAACGACCCCCG |
| | CCCATTGACGTCAATAATGACGTATGTTCCCATAGTAACGCCAATAGGG |
| | ACTTTCCATTGACGTCAATGGGTGGAGTATTTACGGTAAACTGCCCACT |
| | TGGCAGTACATCAAGTGTATCATATGCCAAGTACGCCCCCTATTGACGT |
| | CAATGACGGTAAATGGCCCGCCTGGCATTATGCCCAGTACATGACCTTA |
| | TGGGACTTTCCTACTTGGCAGTACATCTACGTATTAGTCATCGCTATTA |
| | CCATGGTGATGCGGTTTTGGCAGTACATCAATGGGCGTGGATAGCGGTT |
| | TGACTCACGGGGATTTCCAAGTCTCCACCCCATTGACGTCAATGGGAGT |
| | TTGTTTTGGCACCAAAATCAACGGGACTTTCCAAAATGTCGTAACAACT |
| | CCGCCCCATTGACGCAAATGGGCGGTAGGCGTGTACGGTGGGAGGTCTA |
| | TATAAGCAGAGCTGGTTTAGTGAACCGTCAGATCCTGCAGAAGTTGGTC |
| | GTGAGGCACTGGGCAGGTAAGTATCAAGGTTACAAGACAGGTTTAAGGA |
| | GACCAATAGAAACTGGGCTTGTCGAGACAGAGAAGACTCTTGCGTTTCT |
| | GATAGGCACCTATTGGTCTTACTGACATCCACTTTGCCTTTCTCTCCAC |
| | AGGTGTCCAGGCGGCCGCCATGGTGAGCAAGGGCGAGGAGCTGTTCACC |
| | GGGGTGGTGCCCATCCTGGTCGAGCTGGACGGCGACGTAAACGGCCACA |
| | AGTTCAGCGTGTCCGGCGAGGGCGAGGGCGATGCCACCTACGGCAAGCT |
| | GACCCTGAAGTTCATCTGCACCACCGGCAAGCTGCCCGTGCCCTGGCCC |
| | ACCCTCGTGACCACCCTGACCTACGGCGTGCAGTGCTTCAGCCGCTACC |
| | CCGACCACATGAAGCAGCACGACTTCTTCAAGTCCGCCATGCCCGAAGG |
| | CTACGTCCAGGAGCGCACCATCTTCTTCAAGGACGACGGCAACTACAAG |
| | ACCCGCGCCGAGGTGAAGTTCGAGGGCGACACCCTGGTGAACCGCATCG |
| | AGCTGAAGGGCATCGACTTCAAGGAGGACGGCAACATCCTGGGGCACAA |
| | GCTGGAGTACAACTACAACAGCCACAACGTCTATATCATGGCCGACAAG |
| | CAGAAGAACGGCATCAAGGTGAACTTCAAGATCCGCCACAACATCGAGG |
| | ACGGCAGCGTGCAGCTCGCCGACCACTACCAGCAGAACACCCCCATCGG |
| | CGACGGCCCCGTGCTGCTGCCCGACAACCACTACCTGAGCACCCAGTCC |
| | GCCCTGAGCAAAGACCCCAACGAGAAGCGCGATCACATGGTCCTGCTGG |
| | AGTTCGTGACCGCCGCCGGGATCACTCTCGGCATGGACGAGCTGTACAA |
| | GTAATAAGCTTGGATCCAATCAACCTCTGGATTACAAAATTTGTGAAAG |
| | ATTGACTGGTATTCTTAACTATGTTGCTCCTTTTACGCTATGTGGATAC |
| | GCTGCTTTAATGCCTTTGTATCATGCTATTGCTTCCCGTATGGCTTTCA |
| | TTTTCTCCTCCTTGTATAAATCCTGGTTGCTGTCTCTTTATGAGGAGTT |
| | GTGGCCCGTTGTCAGGCAACGTGGCGTGGTGTGCACTGTGTTTGCTGAC |
| | GCAACCCCCACTGGTTGGGGCATTGCCACCACCTGTCAGCTCCTTTCCG |
| | GGACTTTCGCTTTCCCCCTCCCTATTGCCACGGCGGAACTCATCGCCGC |
| | CTGCCTTGCCCGCTGCTGGACAGGGGCTCGGCTGTTGGGCACTGACAAT |
| | TCCGTGGTGTTGTCGGGGAAATCATCGTCCTTTCCTTGGCTGCTCGCCT |
| | GTGTTGCCACCTGGATTCTGCGCGGGACGTCCTTCTGCTACGTCCCTTC |
| | GGCCCTCAATCCAGCGGACCTTCCTTCCCGCGGCCTGCTGCCGGCTCTG |
| | CGGCCTCTTCCGCGTCTTCGAGATCTGCCTCGACTGTGCCTTCTAGTTG |
| | CCAGCCATCTGTTGTTTGCCCCTCCCCCGTGCCTTCCTTGACCCTGGAA |
| | GGTGCCACTCCCACTGTCCTTTCCTAATAAAATGAGGAAATTGCATCGC |
| | ATTGTCTGAGTAGGTGTCATTCTATTCTGGGGGGTGGGGGGGGCAGGAC |
| | AGCAAGGGGGAGGATTGGGAAGACAATAGCAGGCATGCTGGGGACTCGA |
| | ACTGGTGTACCACAGCGTTTCGAGTTAAGGGCGAATTCCCGATAAGGAT |
| | CTTCCTAGAGCATGGCTACGTAGATAAGTAGCATGGCGGGTTAATCATT |
| | AACTACAAGGAACCCCTAGTGATGGAGTTGGCCACTCCCTCTCTGCGCG |
| | CTCGCTCGCTCACTGAGGCCGGGCGACCAAAGGTCGCCCGACGCCCGGG |
| | CTTTGCCCGGGCGGCCTCAGTGAGCGAGCGAGCGCGCAG |
| 32 | rAAV with AAV CTCF divergent sequence knocked |
| | in on positive sense strand |
| | CCGCTCGCTCGCTCACTGAGGCCGCCCGGGCAAAGCCCGGGCGTCGGGC |
| | GACCTTTGGTCGCCCGGCCTCAGTGAGCGAGCGAGCGCGCAGAGAGGGA |
| | GTGGCCAACTCCATCACTAGGGGTTCCTTGTAGTTAATGATTAACCCGC |
| | CATGCTACTTATCTACGTAGCCATGCTCTAGGAAGATCGGAATTCGCCC |
| | TTAAGCTAGTTGCGACACCATGTGGTCACTAGCTAGTTATTAATAGTAA |
| | TCAATTACGGGGTCATTAGTTCATAGCCCATATATGGAGTTCCGCGTTA |
| | CATAACTTACGGTAAATGGCCCGCCTGGCTGACCGCCCAACGACCCCCG |
| | CCCATTGACGTCAATAATGACGTATGTTCCCATAGTAACGCCAATAGGG |
| | ACTTTCCATTGACGTCAATGGGTGGAGTATTTACGGTAAACTGCCCACT |
| | TGGCAGTACATCAAGTGTATCATATGCCAAGTACGCCCCCTATTGACGT |
| | CAATGACGGTAAATGGCCCGCCTGGCATTATGCCCAGTACATGACCTTA |
| | TGGGACTTTCCTACTTGGCAGTACATCTACGTATTAGTCATCGCTATTA |
| | CCATGGTGATGCGGTTTTGGCAGTACATCAATGGGCGTGGATAGCGGTT |
| | TGACTCACGGGGATTTCCAAGTCTCCACCCCATTGACGTCAATGGGAGT |
| | TTGTTTTGGCACCAAAATCAACGGGACTTTCCAAAATGTCGTAACAACT |
| | CCGCCCCATTGACGCAAATGGGCGGTAGGCGTGTACGGTGGGAGGTCTA |
| | TATAAGCAGAGCTGGTTTAGTGAACCGTCAGATCCTGCAGAAGTTGGTC |
| | GTGAGGCACTGGGCAGGTAAGTATCAAGGTTACAAGACAGGTTTAAGGA |
| | GACCAATAGAAACTGGGCTTGTCGAGACAGAGAAGACTCTTGCGTTTCT |
| | GATAGGCACCTATTGGTCTTACTGACATCCACTTTGCCTTTCTCTCCAC |
| | AGGTGTCCAGGCGGCCGCCATGGTGAGCAAGGGCGAGGAGCTGTTCACC |
| | GGGGTGGTGCCCATCCTGGTCGAGCTGGACGGCGACGTAAACGGCCACA |
| | AGTTCAGCGTGTCCGGCGAGGGCGAGGGCGATGCCACCTACGGCAAGCT |
| | GACCCTGAAGTTCATCTGCACCACCGGCAAGCTGCCCGTGCCCTGGCCC |
| | ACCCTCGTGACCACCCTGACCTACGGCGTGCAGTGCTTCAGCCGCTACC |
| | CCGACCACATGAAGCAGCACGACTTCTTCAAGTCCGCCATGCCCGAAGG |
| | CTACGTCCAGGAGCGCACCATCTTCTTCAAGGACGACGGCAACTACAAG |
| | ACCCGCGCCGAGGTGAAGTTCGAGGGCGACACCCTGGTGAACCGCATCG |
| | AGCTGAAGGGCATCGACTTCAAGGAGGACGGCAACATCCTGGGGCACAA |
| | GCTGGAGTACAACTACAACAGCCACAACGTCTATATCATGGCCGACAAG |
| | CAGAAGAACGGCATCAAGGTGAACTTCAAGATCCGCCACAACATCGAGG |
| | ACGGCAGCGTGCAGCTCGCCGACCACTACCAGCAGAACACCCCCATCGG |
| | CGACGGCCCCGTGCTGCTGCCCGACAACCACTACCTGAGCACCCAGTCC |
| | GCCCTGAGCAAAGACCCCAACGAGAAGCGCGATCACATGGTCCTGCTGG |
| | AGTTCGTGACCGCCGCCGGGATCACTCTCGGCATGGACGAGCTGTACAA |
| | GTAATAAGCTTGGATCCAATCAACCTCTGGATTACAAAATTTGTGAAAG |
| | ATTGACTGGTATTCTTAACTATGTTGCTCCTTTTACGCTATGTGGATAC |
| | GCTGCTTTAATGCCTTTGTATCATGCTATTGCTTCCCGTATGGCTTTCA |
| | TTTTCTCCTCCTTGTATAAATCCTGGTTGCTGTCTCTTTATGAGGAGTT |
| | GTGGCCCGTTGTCAGGCAACGTGGCGTGGTGTGCACTGTGTTTGCTGAC |
| | GCAACCCCCACTGGTTGGGGCATTGCCACCACCTGTCAGCTCCTTTCCG |
| | GGACTTTCGCTTTCCCCCTCCCTATTGCCACGGCGGAACTCATCGCCGC |
| | CTGCCTTGCCCGCTGCTGGACAGGGGCTCGGCTGTTGGGCACTGACAAT |
| | TCCGTGGTGTTGTCGGGGAAATCATCGTCCTTTCCTTGGCTGCTCGCCT |
| | GTGTTGCCACCTGGATTCTGCGCGGGACGTCCTTCTGCTACGTCCCTTC |
| | GGCCCTCAATCCAGCGGACCTTCCTTCCCGCGGCCTGCTGCCGGCTCTG |
| | CGGCCTCTTCCGCGTCTTCGAGATCTGCCTCGACTGTGCCTTCTAGTTG |
| | CCAGCCATCTGTTGTTTGCCCCTCCCCCGTGCCTTCCTTGACCCTGGAA |
| | GGTGCCACTCCCACTGTCCTTTCCTAATAAAATGAGGAAATTGCATCGC |
| | ATTGTCTGAGTAGGTGTCATTCTATTCTGGGGGGTGGGGTGGGGCAGGA |
| | CAGCAAGGGGGAGGATTGGGAAGACAATAGCAGGCATGCTGGGGACTCG |
| | ATTGCGACACCATGTGGTCATCGAGTTAAGGGCGAATTCCCGATAAGGA |
| | TCTTCCTAGAGCATGGCTACGTAGATAAGTAGCATGGCGGGTTAATCAT |
| | TAACTACAAGGAACCCCTAGTGATGGAGTTGGCCACTCCCTCTCTGCGC |
| | GCTCGCTCGCTCACTGAGGCCGGGCGACCAAAGGTCGCCCGACGCCCGG |
| | GCTTTGCCCGGGCGGCCTCAGTGAGCGAGCGAGCGCGCAG |
| 33 | rAAV with AAV CTCF repeat convergent sequence |
| | knocked in on positive-sense strand |
| | CCGCTCGCTCGCTCACTGAGGCCGCCCGGGCAAAGCCCGGGCGTCGGGC |
| | GACCTTTGGTCGCCCGGCCTCAGTGAGCGAGCGAGCGCGCAGAGAGGGA |
| | GTGGCCAACTCCATCACTAGGGGTTCCTTGTAGTTAATGATTAACCCGC |
| | CATGCTACTTATCTACGTAGCCATGCTCTAGGAAGATCGGAATTCGCCC |
| | TTAAGCTAGTTGCGACACCATGTGGTCATTGCGACACCATGTGGTCATT |
| | GCGACACCATGTGGTCATTGCGACACCATGTGGTCATTGCGACACCATG |
| | TGGTCACTAGCTAGTTATTAATAGTAATCAATTACGGGGTCATTAGTTC |
| | ATAGCCCATATATGGAGTTCCGCGTTACATAACTTACGGTAAATGGCCC |
| | GCCTGGCTGACCGCCCAACGACCCCCGCCCATTGACGTCAATAATGACG |
| | TATGTTCCCATAGTAACGCCAATAGGGACTTTCCATTGACGTCAATGGG |
| | TGGAGTATTTACGGTAAACTGCCCACTTGGCAGTACATCAAGTGTATCA |
| | TATGCCAAGTACGCCCCCTATTGACGTCAATGACGGTAAATGGCCCGCC |
| | TGGCATTATGCCCAGTACATGACCTTATGGGACTTTCCTACTTGGCAGT |
| | ACATCTACGTATTAGTCATCGCTATTACCATGGTGATGCGGTTTTGGCA |
| | GTACATCAATGGGCGTGGATAGCGGTTTGACTCACGGGGATTTCCAAGT |
| | CTCCACCCCATTGACGTCAATGGGAGTTTGTTTTGGCACCAAAATCAAC |
| | GGGACTTTCCAAAATGTCGTAACAACTCCGCCCCATTGACGCAAATGGG |
| | CGGTAGGCGTGTACGGTGGGAGGTCTATATAAGCAGAGCTGGTTTAGTG |
| | AACCGTCAGATCCTGCAGAAGTTGGTCGTGAGGCACTGGGCAGGTAAGT |
| | ATCAAGGTTACAAGACAGGTTTAAGGAGACCAATAGAAACTGGGCTTGT |
| | CGAGACAGAGAAGACTCTTGCGTTTCTGATAGGCACCTATTGGTCTTAC |
| | TGACATCCACTTTGCCTTTCTCTCCACAGGTGTCCAGGCGGCCGCCATG |
| | GTGAGCAAGGGCGAGGAGCTGTTCACCGGGGTGGTGCCCATCCTGGTCG |
| | AGCTGGACGGCGACGTAAACGGCCACAAGTTCAGCGTGTCCGGCGAGGG |
| | CGAGGGCGATGCCACCTACGGCAAGCTGACCCTGAAGTTCATCTGCACC |
| | ACCGGCAAGCTGCCCGTGCCCTGGCCCACCCTCGTGACCACCCTGACCT |
| | ACGGCGTGCAGTGCTTCAGCCGCTACCCCGACCACATGAAGCAGCACGA |
| | CTTCTTCAAGTCCGCCATGCCCGAAGGCTACGTCCAGGAGCGCACCATC |
| | TTCTTCAAGGACGACGGCAACTACAAGACCCGCGCCGAGGTGAAGTTCG |
| | AGGGCGACACCCTGGTGAACCGCATCGAGCTGAAGGGCATCGACTTCAA |
| | GGAGGACGGCAACATCCTGGGGCACAAGCTGGAGTACAACTACAACAGC |
| | CACAACGTCTATATCATGGCCGACAAGCAGAAGAACGGCATCAAGGTGA |
| | ACTTCAAGATCCGCCACAACATCGAGGACGGCAGCGTGCAGCTCGCCGA |
| | CCACTACCAGCAGAACACCCCCATCGGCGACGGCCCCGTGCTGCTGCCC |
| | GACAACCACTACCTGAGCACCCAGTCCGCCCTGAGCAAAGACCCCAACG |
| | AGAAGCGCGATCACATGGTCCTGCTGGAGTTCGTGACCGCCGCCGGGAT |
| | CACTCTCGGCATGGACGAGCTGTACAAGTAATAAGCTTGGATCCAATCA |
| | ACCTCTGGATTACAAAATTTGTGAAAGATTGACTGGTATTCTTAACTAT |
| | GTTGCTCCTTTTACGCTATGTGGATACGCTGCTTTAATGCCTTTGTATC |
| | ATGCTATTGCTTCCCGTATGGCTTTCATTTTCTCCTCCTTGTATAAATC |
| | CTGGTTGCTGTCTCTTTATGAGGAGTTGTGGCCCGTTGTCAGGCAACGT |
| | GGCGTGGTGTGCACTGTGTTTGCTGACGCAACCCCCACTGGTTGGGGCA |
| | TTGCCACCACCTGTCAGCTCCTTTCCGGGACTTTCGCTTTCCCCCTCCC |
| | TATTGCCACGGCGGAACTCATCGCCGCCTGCCTTGCCCGCTGCTGGACA |
| | GGGGCTCGGCTGTTGGGCACTGACAATTCCGTGGTGTTGTCGGGGAAAT |
| | CATCGTCCTTTCCTTGGCTGCTCGCCTGTGTTGCCACCTGGATTCTGCG |
| | CGGGACGTCCTTCTGCTACGTCCCTTCGGCCCTCAATCCAGCGGACCTT |
| | CCTTCCCGCGGCCTGCTGCCGGCTCTGCGGCCTCTTCCGCGTCTTCGAG |
| | ATCTGCCTCGACTGTGCCTTCTAGTTGCCAGCCATCTGTTGTTTGCCCC |
| | TCCCCCGTGCCTTCCTTGACCCTGGAAGGTGCCACTCCCACTGTCCTTT |
| | CCTAATAAAATGAGGAAATTGCATCGCATTGTCTGAGTAGGTGTCATTC |
| | TATTCTGGGGGGTGGGGTGGGGCAGGACAGCAAGGGGGAGGATTGGGAA |
| | GACAATAGCAGGCATGCTGGGGACTCGAACTGGTGTACCACAGCGTTAC |
| | TGGTGTACCACAGCGTTACTGGTGTACCACAGCGTTACTGGTGTACCAC |
| | AGCGTTACTGGTGTACCACAGCGTTTCGAGTTAAGGGCGAATTCCCGAT |
| | AAGGATCTTCCTAGAGCATGGCTACGTAGATAAGTAGCATGGCGGGTTA |
| | ATCATTAACTACAAGGAACCCCTAGTGATGGAGTTGGCCACTCCCTCTC |
| | TGCGCGCTCGCTCGCTCACTGAGGCCGGGCGACCAAAGGTCGCCCGACG |
| | CCCGGGCTTTGCCCGGGCGGCCTCAGTGAGCGAGCGAGCGCGCAG |
| 34 | rAAV with AAV CTCF forward on positive-sense |
| | and reverse on negative-sense strand, convergent |
| | CCGCTCGCTCGCTCACTGAGGCCGCCCGGGCAAAGCCCGGGCGTCGGGC |
| | GACCTTTGGTCGCCCGGCCTCAGTGAGCGAGCGAGCGCGCAGAGAGGGA |
| | GTGGCCAACTCCATCACTAGGGGTTCCTTGTAGTTAATGATTAACCCGC |
| | CATGCTACTTATCTACGTAGCCATGCTCTAGGAAGATCGGAATTCGCCC |
| | TTAAGCTAGTTGCGACACCATGTGGTCACTAGCTAGTTATTAATAGTAA |
| | TCAATTACGGGGTCATTAGTTCATAGCCCATATATGGAGTTCCGCGTTA |
| | CATAACTTACGGTAAATGGCCCGCCTGGCTGACCGCCCAACGACCCCCG |
| | CCCATTGACGTCAATAATGACGTATGTTCCCATAGTAACGCCAATAGGG |
| | ACTTTCCATTGACGTCAATGGGTGGAGTATTTACGGTAAACTGCCCACT |
| | TGGCAGTACATCAAGTGTATCATATGCCAAGTACGCCCCCTATTGACGT |
| | CAATGACGGTAAATGGCCCGCCTGGCATTATGCCCAGTACATGACCTTA |
| | TGGGACTTTCCTACTTGGCAGTACATCTACGTATTAGTCATCGCTATTA |
| | CCATGGTGATGCGGTTTTGGCAGTACATCAATGGGCGTGGATAGCGGTT |
| | TGACTCACGGGGATTTCCAAGTCTCCACCCCATTGACGTCAATGGGAGT |
| | TTGTTTTGGCACCAAAATCAACGGGACTTTCCAAAATGTCGTAACAACT |
| | CCGCCCCATTGACGCAAATGGGCGGTAGGCGTGTACGGTGGGAGGTCTA |
| | TATAAGCAGAGCTGGTTTAGTGAACCGTCAGATCCTGCAGAAGTTGGTC |
| | GTGAGGCACTGGGCAGGTAAGTATCAAGGTTACAAGACAGGTTTAAGGA |
| | GACCAATAGAAACTGGGCTTGTCGAGACAGAGAAGACTCTTGCGTTTCT |
| | GATAGGCACCTATTGGTCTTACTGACATCCACTTTGCCTTTCTCTCCAC |
| | AGGTGTCCAGGCGGCCGCCATGGTGAGCAAGGGCGAGGAGCTGTTCACC |
| | GGGGTGGTGCCCATCCTGGTCGAGCTGGACGGCGACGTAAACGGCCACA |
| | AGTTCAGCGTGTCCGGCGAGGGCGAGGGCGATGCCACCTACGGCAAGCT |
| | GACCCTGAAGTTCATCTGCACCACCGGCAAGCTGCCCGTGCCCTGGCCC |
| | ACCCTCGTGACCACCCTGACCTACGGCGTGCAGTGCTTCAGCCGCTACC |
| | CCGACCACATGAAGCAGCACGACTTCTTCAAGTCCGCCATGCCCGAAGG |
| | CTACGTCCAGGAGCGCACCATCTTCTTCAAGGACGACGGCAACTACAAG |
| | ACCCGCGCCGAGGTGAAGTTCGAGGGCGACACCCTGGTGAACCGCATCG |
| | AGCTGAAGGGCATCGACTTCAAGGAGGACGGCAACATCCTGGGGCACAA |
| | GCTGGAGTACAACTACAACAGCCACAACGTCTATATCATGGCCGACAAG |
| | CAGAAGAACGGCATCAAGGTGAACTTCAAGATCCGCCACAACATCGAGG |
| | ACGGCAGCGTGCAGCTCGCCGACCACTACCAGCAGAACACCCCCATCGG |
| | CGACGGCCCCGTGCTGCTGCCCGACAACCACTACCTGAGCACCCAGTCC |
| | GCCCTGAGCAAAGACCCCAACGAGAAGCGCGATCACATGGTCCTGCTGG |
| | AGTTCGTGACCGCCGCCGGGATCACTCTCGGCATGGACGAGCTGTACAA |
| | GTAATAAGCTTGGATCCAATCAACCTCTGGATTACAAAATTTGTGAAAG |
| | ATTGACTGGTATTCTTAACTATGTTGCTCCTTTTACGCTATGTGGATAC |
| | GCTGCTTTAATGCCTTTGTATCATGCTATTGCTTCCCGTATGGCTTTCA |
| | TTTTCTCCTCCTTGTATAAATCCTGGTTGCTGTCTCTTTATGAGGAGTT |
| | GTGGCCCGTTGTCAGGCAACGTGGCGTGGTGTGCACTGTGTTTGCTGAC |
| | GCAACCCCCACTGGTTGGGGCATTGCCACCACCTGTCAGCTCCTTTCCG |
| | GGACTTTCGCTTTCCCCCTCCCTATTGCCACGGCGGAACTCATCGCCGC |
| | CTGCCTTGCCCGCTGCTGGACAGGGGCTCGGCTGTTGGGCACTGACAAT |
| | TCCGTGGTGTTGTCGGGGAAATCATCGTCCTTTCCTTGGCTGCTCGCCT |
| | GTGTTGCCACCTGGATTCTGCGCGGGACGTCCTTCTGCTACGTCCCTTC |
| | GGCCCTCAATCCAGCGGACCTTCCTTCCCGCGGCCTGCTGCCGGCTCTG |
| | CGGCCTCTTCCGCGTCTTCGAGATCTGCCTCGACTGTGCCTTCTAGTTG |
| | CCAGCCATCTGTTGTTTGCCCCTCCCCCGTGCCTTCCTTGACCCTGGAA |
| | GGTGCCACTCCCACTGTCCTTTCCTAATAAAATGAGGAAATTGCATCGC |
| | ATTGTCTGAGTAGGTGTCATTCTATTCTGGGGGGTGGGGTGGGGCAGGA |
| | CAGCAAGGGGGAGGATTGGGAAGACAATAGCAGGCATGCTGGGGACTCG |
| | ATGACCACATGGTGTCGCAATCGAGTTAAGGGCGAATTCCCGATAAGGA |
| | TCTTCCTAGAGCATGGCTACGTAGATAAGTAGCATGGCGGGTTAATCAT |
| | TAACTACAAGGAACCCCTAGTGATGGAGTTGGCCACTCCCTCTCTGCGC |
| | GCTCGCTCGCTCACTGAGGCCGGGCGACCAAAGGTCGCCCGACGCCCGG |
| | GCTTTGCCCGGGCGGCCTCAGTGAGCGAGCGAGCGCGCAG |
| 35 | rAAV with forward human CTCF sequence knocked |
| | in on positive-sense strand |
| | CTGCGCGCTCGCTCGCTCACTGAGGCCGCCCGGGCAAAGCCCGGGCGTC |
| | GGGCGACCTTTGGTCGCCCGGCCTCAGTGAGCGAGCGAGCGCGCAGAGA |
| | GGGAGTGGCCAACTCCATCACTAGGGGTTCCTTGTAGTTAATGATTAAC |
| | CCGCCATGCTACTTATCTACGTAGCCATGCTCTAGGAAGATCGGAATTC |
| | GCCCTTAAGCTAGCCACAAGGTGGCGCCTAGCTAGTTATTAATAGTAAT |
| | CAATTACGGGGTCATTAGTTCATAGCCCATATATGGAGTTCCGCGTTAC |
| | ATAACTTACGGTAAATGGCCCGCCTGGCTGACCGCCCAACGACCCCCGC |
| | CCATTGACGTCAATAATGACGTATGTTCCCATAGTAACGCCAATAGGGA |
| | CTTTCCATTGACGTCAATGGGTGGAGTATTTACGGTAAACTGCCCACTT |
| | GGCAGTACATCAAGTGTATCATATGCCAAGTACGCCCCCTATTGACGTC |
| | AATGACGGTAAATGGCCCGCCTGGCATTATGCCCAGTACATGACCTTAT |
| | GGGACTTTCCTACTTGGCAGTACATCTACGTATTAGTCATCGCTATTAC |
| | CATGGTGATGCGGTTTTGGCAGTACATCAATGGGCGTGGATAGCGGTTT |
| | GACTCACGGGGATTTCCAAGTCTCCACCCCATTGACGTCAATGGGAGTT |
| | TGTTTTGGCACCAAAATCAACGGGACTTTCCAAAATGTCGTAACAACTC |
| | CGCCCCATTGACGCAAATGGGCGGTAGGCGTGTACGGTGGGAGGTCTAT |
| | ATAAGCAGAGCTGGTTTAGTGAACCGTCAGATCCTGCAGAAGTTGGTCG |
| | TGAGGCACTGGGCAGGTAAGTATCAAGGTTACAAGACAGGTTTAAGGAG |
| | ACCAATAGAAACTGGGCTTGTCGAGACAGAGAAGACTCTTGCGTTTCTG |
| | ATAGGCACCTATTGGTCTTACTGACATCCACTTTGCCTTTCTCTCCACA |
| | GGTGTCCAGGCGGCCGCCATGGTGAGCAAGGGCGAGGAGCTGTTCACCG |
| | GGGTGGTGCCCATCCTGGTCGAGCTGGACGGCGACGTAAACGGCCACAA |
| | GTTCAGCGTGTCCGGCGAGGGCGAGGGCGATGCCACCTACGGCAAGCTG |
| | ACCCTGAAGTTCATCTGCACCACCGGCAAGCTGCCCGTGCCCTGGCCCA |
| | CCCTCGTGACCACCCTGACCTACGGCGTGCAGTGCTTCAGCCGCTACCC |
| | CGACCACATGAAGCAGCACGACTTCTTCAAGTCCGCCATGCCCGAAGGC |
| | TACGTCCAGGAGCGCACCATCTTCTTCAAGGACGACGGCAACTACAAGA |
| | CCCGCGCCGAGGTGAAGTTCGAGGGCGACACCCTGGTGAACCGCATCGA |
| | GCTGAAGGGCATCGACTTCAAGGAGGACGGCAACATCCTGGGGCACAAG |
| | CTGGAGTACAACTACAACAGCCACAACGTCTATATCATGGCCGACAAGC |
| | AGAAGAACGGCATCAAGGTGAACTTCAAGATCCGCCACAACATCGAGGA |
| | CGGCAGCGTGCAGCTCGCCGACCACTACCAGCAGAACACCCCCATCGGC |
| | GACGGCCCCGTGCTGCTGCCCGACAACCACTACCTGAGCACCCAGTCCG |
| | CCCTGAGCAAAGACCCCAACGAGAAGCGCGATCACATGGTCCTGCTGGA |
| | GTTCGTGACCGCCGCCGGGATCACTCTCGGCATGGACGAGCTGTACAAG |
| | TAATAAGCTTGGATCCAATCAACCTCTGGATTACAAAATTTGTGAAAGA |
| | TTGACTGGTATTCTTAACTATGTTGCTCCTTTTACGCTATGTGGATACG |
| | CTGCTTTAATGCCTTTGTATCATGCTATTGCTTCCCGTATGGCTTTCAT |
| | TTTCTCCTCCTTGTATAAATCCTGGTTGCTGTCTCTTTATGAGGAGTTG |
| | TGGCCCGTTGTCAGGCAACGTGGCGTGGTGTGCACTGTGTTTGCTGACG |
| | CAACCCCCACTGGTTGGGGCATTGCCACCACCTGTCAGCTCCTTTCCGG |
| | GACTTTCGCTTTCCCCCTCCCTATTGCCACGGCGGAACTCATCGCCGCC |
| | TGCCTTGCCCGCTGCTGGACAGGGGCTCGGCTGTTGGGCACTGACAATT |
| | CCGTGGTGTTGTCGGGGAAATCATCGTCCTTTCCTTGGCTGCTCGCCTG |
| | TGTTGCCACCTGGATTCTGCGCGGGACGTCCTTCTGCTACGTCCCTTCG |
| | GCCCTCAATCCAGCGGACCTTCCTTCCCGCGGCCTGCTGCCGGCTCTGC |
| | GGCCTCTTCCGCGTCTTCGAGATCTGCCTCGACTGTGCCTTCTAGTTGC |
| | CAGCCATCTGTTGTTTGCCCCTCCCCCGTGCCTTCCTTGACCCTGGAAG |
| | GTGCCACTCCCACTGTCCTTTCCTAATAAAATGAGGAAATTGCATCGCA |
| | TTGTCTGAGTAGGTGTCATTCTATTCTGGGGGGTGGGGTGGGGCAGGAC |
| | AGCAAGGGGGAGGATTGGGAAGACAATAGCAGGCATGCTGGGGACTCGA |
| | TCGAGTTAAGGGCGAATTCCCGATAAGGATCTTCCTAGAGCATGGCTAC |
| | GTAGATAAGTAGCATGGCGGGTTAATCATTAACTACAAGGAACCCCTAG |
| | TGATGGAGTTGGCCACTCCCTCTCTGCGCGCTCGCTCGCTCACTGAGGC |
| | CGGGCGACCAAAGGTCGCCCGACGCCCGGGCTTTGCCCGGGCGGCCTCA |
| | GTGAGCGAGCGAGCGCGCAG |
| 36 | rAAV with reverse human CTCF sequence knocked |
| | in on positive-sense strand |
| | CTGCGCGCTCGCTCGCTCACTGAGGCCGCCCGGGCAAAGCCCGGGCGTC |
| | GGGCGACCTTTGGTCGCCCGGCCTCAGTGAGCGAGCGAGCGCGCAGAGA |
| | GGGAGTGGCCAACTCCATCACTAGGGGTTCCTTGTAGTTAATGATTAAC |
| | CCGCCATGCTACTTATCTACGTAGCCATGCTCTAGGAAGATCGGAATTC |
| | GCCCTTAAGCTAGCTAGCTAGTTATTAATAGTAATCAATTACGGGGTCA |
| | TTAGTTCATAGCCCATATATGGAGTTCCGCGTTACATAACTTACGGTAA |
| | ATGGCCCGCCTGGCTGACCGCCCAACGACCCCCGCCCATTGACGTCAAT |
| | AATGACGTATGTTCCCATAGTAACGCCAATAGGGACTTTCCATTGACGT |
| | CAATGGGTGGAGTATTTACGGTAAACTGCCCACTTGGCAGTACATCAAG |
| | TGTATCATATGCCAAGTACGCCCCCTATTGACGTCAATGACGGTAAATG |
| | GCCCGCCTGGCATTATGCCCAGTACATGACCTTATGGGACTTTCCTACT |
| | TGGCAGTACATCTACGTATTAGTCATCGCTATTACCATGGTGATGCGGT |
| | TTTGGCAGTACATCAATGGGCGTGGATAGCGGTTTGACTCACGGGGATT |
| | TCCAAGTCTCCACCCCATTGACGTCAATGGGAGTTTGTTTTGGCACCAA |
| | AATCAACGGGACTTTCCAAAATGTCGTAACAACTCCGCCCCATTGACGC |
| | AAATGGGCGGTAGGCGTGTACGGTGGGAGGTCTATATAAGCAGAGCTGG |
| | TTTAGTGAACCGTCAGATCCTGCAGAAGTTGGTCGTGAGGCACTGGGCA |
| | GGTAAGTATCAAGGTTACAAGACAGGTTTAAGGAGACCAATAGAAACTG |
| | GGCTTGTCGAGACAGAGAAGACTCTTGCGTTTCTGATAGGCACCTATTG |
| | GTCTTACTGACATCCACTTTGCCTTTCTCTCCACAGGTGTCCAGGCGGC |
| | CGCCATGGTGAGCAAGGGCGAGGAGCTGTTCACCGGGGTGGTGCCCATC |
| | CTGGTCGAGCTGGACGGCGACGTAAACGGCCACAAGTTCAGCGTGTCCG |
| | GCGAGGGCGAGGGCGATGCCACCTACGGCAAGCTGACCCTGAAGTTCAT |
| | CTGCACCACCGGCAAGCTGCCCGTGCCCTGGCCCACCCTCGTGACCACC |
| | CTGACCTACGGCGTGCAGTGCTTCAGCCGCTACCCCGACCACATGAAGC |
| | AGCACGACTTCTTCAAGTCCGCCATGCCCGAAGGCTACGTCCAGGAGCG |
| | CACCATCTTCTTCAAGGACGACGGCAACTACAAGACCCGCGCCGAGGTG |
| | AAGTTCGAGGGCGACACCCTGGTGAACCGCATCGAGCTGAAGGGCATCG |
| | ACTTCAAGGAGGACGGCAACATCCTGGGGCACAAGCTGGAGTACAACTA |
| | CAACAGCCACAACGTCTATATCATGGCCGACAAGCAGAAGAACGGCATC |
| | AAGGTGAACTTCAAGATCCGCCACAACATCGAGGACGGCAGCGTGCAGC |
| | TCGCCGACCACTACCAGCAGAACACCCCCATCGGCGACGGCCCCGTGCT |
| | GCTGCCCGACAACCACTACCTGAGCACCCAGTCCGCCCTGAGCAAAGAC |
| | CCCAACGAGAAGCGCGATCACATGGTCCTGCTGGAGTTCGTGACCGCCG |
| | CCGGGATCACTCTCGGCATGGACGAGCTGTACAAGTAATAAGCTTGGAT |
| | CCAATCAACCTCTGGATTACAAAATTTGTGAAAGATTGACTGGTATTCT |
| | TAACTATGTTGCTCCTTTTACGCTATGTGGATACGCTGCTTTAATGCCT |
| | TTGTATCATGCTATTGCTTCCCGTATGGCTTTCATTTTCTCCTCCTTGT |
| | ATAAATCCTGGTTGCTGTCTCTTTATGAGGAGTTGTGGCCCGTTGTCAG |
| | GCAACGTGGCGTGGTGTGCACTGTGTTTGCTGACGCAACCCCCACTGGT |
| | TGGGGCATTGCCACCACCTGTCAGCTCCTTTCCGGGACTTTCGCTTTCC |
| | CCCTCCCTATTGCCACGGCGGAACTCATCGCCGCCTGCCTTGCCCGCTG |
| | CTGGACAGGGGCTCGGCTGTTGGGCACTGACAATTCCGTGGTGTTGTCG |
| | GGGAAATCATCGTCCTTTCCTTGGCTGCTCGCCTGTGTTGCCACCTGGA |
| | TTCTGCGCGGGACGTCCTTCTGCTACGTCCCTTCGGCCCTCAATCCAGC |
| | GGACCTTCCTTCCCGCGGCCTGCTGCCGGCTCTGCGGCCTCTTCCGCGT |
| | CTTCGAGATCTGCCTCGACTGTGCCTTCTAGTTGCCAGCCATCTGTTGT |
| | TTGCCCCTCCCCCGTGCCTTCCTTGACCCTGGAAGGTGCCACTCCCACT |
| | GTCCTTTCCTAATAAAATGAGGAAATTGCATCGCATTGTCTGAGTAGGT |
| | GTCATTCTATTCTGGGGGGTGGGGTGGGGCAGGACAGCAAGGGGGAGGA |
| | TTGGGAAGACAATAGCAGGCATGCTGGGGACTCGACCGCCCCCTGGTGG |
| | TCGAGTTAAGGGCGAATTCCCGATAAGGATCTTCCTAGAGCATGGCTAC |
| | GTAGATAAGTAGCATGGCGGGTTAATCATTAACTACAAGGAACCCCTAG |
| | TGATGGAGTTGGCCACTCCCTCTCTGCGCGCTCGCTCGCTCACTGAGGC |
| | CGGGCGACCAAAGGTCGCCCGACGCCCGGGCTTTGCCCGGGCGGCCTCA |
| | GTGAGCGAGCGAGCGCGCAG |
| 37 | rAAV with human CTCF convergent sequence |
| | knocked in on positive-sense strand |
| | CTGCGCGCTCGCTCGCTCACTGAGGCCGCCCGGGCAAAGCCCGGGCGTC |
| | GGGCGACCTTTGGTCGCCCGGCCTCAGTGAGCGAGCGAGCGCGCAGAGA |
| | GGGAGTGGCCAACTCCATCACTAGGGGTTCCTTGTAGTTAATGATTAAC |
| | CCGCCATGCTACTTATCTACGTAGCCATGCTCTAGGAAGATCGGAATTC |
| | GCCCTTAAGCTAGCCACAAGGTGGCGCCTAGCTAGTTATTAATAGTAAT |
| | CAATTACGGGGTCATTAGTTCATAGCCCATATATGGAGTTCCGCGTTAC |
| | ATAACTTACGGTAAATGGCCCGCCTGGCTGACCGCCCAACGACCCCCGC |
| | CCATTGACGTCAATAATGACGTATGTTCCCATAGTAACGCCAATAGGGA |
| | CTTTCCATTGACGTCAATGGGTGGAGTATTTACGGTAAACTGCCCACTT |
| | GGCAGTACATCAAGTGTATCATATGCCAAGTACGCCCCCTATTGACGTC |
| | AATGACGGTAAATGGCCCGCCTGGCATTATGCCCAGTACATGACCTTAT |
| | GGGACTTTCCTACTTGGCAGTACATCTACGTATTAGTCATCGCTATTAC |
| | CATGGTGATGCGGTTTTGGCAGTACATCAATGGGCGTGGATAGCGGTTT |
| | GACTCACGGGGATTTCCAAGTCTCCACCCCATTGACGTCAATGGGAGTT |
| | TGTTTTGGCACCAAAATCAACGGGACTTTCCAAAATGTCGTAACAACTC |
| | CGCCCCATTGACGCAAATGGGCGGTAGGCGTGTACGGTGGGAGGTCTAT |
| | ATAAGCAGAGCTGGTTTAGTGAACCGTCAGATCCTGCAGAAGTTGGTCG |
| | TGAGGCACTGGGCAGGTAAGTATCAAGGTTACAAGACAGGTTTAAGGAG |
| | ACCAATAGAAACTGGGCTTGTCGAGACAGAGAAGACTCTTGCGTTTCTG |
| | ATAGGCACCTATTGGTCTTACTGACATCCACTTTGCCTTTCTCTCCACA |
| | GGTGTCCAGGCGGCCGCCATGGTGAGCAAGGGCGAGGAGCTGTTCACCG |
| | GGGTGGTGCCCATCCTGGTCGAGCTGGACGGCGACGTAAACGGCCACAA |
| | GTTCAGCGTGTCCGGCGAGGGCGAGGGCGATGCCACCTACGGCAAGCTG |
| | ACCCTGAAGTTCATCTGCACCACCGGCAAGCTGCCCGTGCCCTGGCCCA |
| | CCCTCGTGACCACCCTGACCTACGGCGTGCAGTGCTTCAGCCGCTACCC |
| | CGACCACATGAAGCAGCACGACTTCTTCAAGTCCGCCATGCCCGAAGGC |
| | TACGTCCAGGAGCGCACCATCTTCTTCAAGGACGACGGCAACTACAAGA |
| | CCCGCGCCGAGGTGAAGTTCGAGGGCGACACCCTGGTGAACCGCATCGA |
| | GCTGAAGGGCATCGACTTCAAGGAGGACGGCAACATCCTGGGGCACAAG |
| | CTGGAGTACAACTACAACAGCCACAACGTCTATATCATGGCCGACAAGC |
| | AGAAGAACGGCATCAAGGTGAACTTCAAGATCCGCCACAACATCGAGGA |
| | CGGCAGCGTGCAGCTCGCCGACCACTACCAGCAGAACACCCCCATCGGC |
| | GACGGCCCCGTGCTGCTGCCCGACAACCACTACCTGAGCACCCAGTCCG |
| | CCCTGAGCAAAGACCCCAACGAGAAGCGCGATCACATGGTCCTGCTGGA |
| | GTTCGTGACCGCCGCCGGGATCACTCTCGGCATGGACGAGCTGTACAAG |
| | TAATAAGCTTGGATCCAATCAACCTCTGGATTACAAAATTTGTGAAAGA |
| | TTGACTGGTATTCTTAACTATGTTGCTCCTTTTACGCTATGTGGATACG |
| | CTGCTTTAATGCCTTTGTATCATGCTATTGCTTCCCGTATGGCTTTCAT |
| | TTTCTCCTCCTTGTATAAATCCTGGTTGCTGTCTCTTTATGAGGAGTTG |
| | TGGCCCGTTGTCAGGCAACGTGGCGTGGTGTGCACTGTGTTTGCTGACG |
| | CAACCCCCACTGGTTGGGGCATTGCCACCACCTGTCAGCTCCTTTCCGG |
| | GACTTTCGCTTTCCCCCTCCCTATTGCCACGGCGGAACTCATCGCCGCC |
| | TGCCTTGCCCGCTGCTGGACAGGGGCTCGGCTGTTGGGCACTGACAATT |
| | CCGTGGTGTTGTCGGGGAAATCATCGTCCTTTCCTTGGCTGCTCGCCTG |
| | TGTTGCCACCTGGATTCTGCGCGGGACGTCCTTCTGCTACGTCCCTTCG |
| | GCCCTCAATCCAGCGGACCTTCCTTCCCGCGGCCTGCTGCCGGCTCTGC |
| | GGCCTCTTCCGCGTCTTCGAGATCTGCCTCGACTGTGCCTTCTAGTTGC |
| | CAGCCATCTGTTGTTTGCCCCTCCCCCGTGCCTTCCTTGACCCTGGAAG |
| | GTGCCACTCCCACTGTCCTTTCCTAATAAAATGAGGAAATTGCATCGCA |
| | TTGTCTGAGTAGGTGTCATTCTATTCTGGGGGGTGGGGTGGGGCAGGAC |
| | AGCAAGGGGGAGGATTGGGAAGACAATAGCAGGCATGCTGGGGACTCGA |
| | CCGCCCCCTGGTGGTCGAGTTAAGGGCGAATTCCCGATAAGGATCTTCC |
| | TAGAGCATGGCTACGTAGATAAGTAGCATGGCGGGTTAATCATTAACTA |
| | CAAGGAACCCCTAGTGATGGAGTTGGCCACTCCCTCTCTGCGCGCTCGC |
| | TCGCTCACTGAGGCCGGGCGACCAAAGGTCGCCCGACGCCCGGGCTTTG |
| | CCCGGGCGGCCTCAGTGAGCGAGCGAGCGCGCAG |
| 38 | rAAV with human CTCF divergent sequence |
| | knocked in on positive sense strand |
| | CTGCGCGCTCGCTCGCTCACTGAGGCCGCCCGGGCAAAGCCCGGGCGTC |
| | GGGCGACCTTTGGTCGCCCGGCCTCAGTGAGCGAGCGAGCGCGCAGAGA |
| | GGGAGTGGCCAACTCCATCACTAGGGGTTCCTTGTAGTTAATGATTAAC |
| | CCGCCATGCTACTTATCTACGTAGCCATGCTCTAGGAAGATCGGAATTC |
| | GCCCTTAAGCTAGCCACAAGGTGGCGCCTAGCTAGTTATTAATAGTAAT |
| | CAATTACGGGGTCATTAGTTCATAGCCCATATATGGAGTTCCGCGTTAC |
| | ATAACTTACGGTAAATGGCCCGCCTGGCTGACCGCCCAACGACCCCCGC |
| | CCATTGACGTCAATAATGACGTATGTTCCCATAGTAACGCCAATAGGGA |
| | CTTTCCATTGACGTCAATGGGTGGAGTATTTACGGTAAACTGCCCACTT |
| | GGCAGTACATCAAGTGTATCATATGCCAAGTACGCCCCCTATTGACGTC |
| | AATGACGGTAAATGGCCCGCCTGGCATTATGCCCAGTACATGACCTTAT |
| | GGGACTTTCCTACTTGGCAGTACATCTACGTATTAGTCATCGCTATTAC |
| | CATGGTGATGCGGTTTTGGCAGTACATCAATGGGCGTGGATAGCGGTTT |
| | GACTCACGGGGATTTCCAAGTCTCCACCCCATTGACGTCAATGGGAGTT |
| | TGTTTTGGCACCAAAATCAACGGGACTTTCCAAAATGTCGTAACAACTC |
| | CGCCCCATTGACGCAAATGGGCGGTAGGCGTGTACGGTGGGAGGTCTAT |
| | ATAAGCAGAGCTGGTTTAGTGAACCGTCAGATCCTGCAGAAGTTGGTCG |
| | TGAGGCACTGGGCAGGTAAGTATCAAGGTTACAAGACAGGTTTAAGGAG |
| | ACCAATAGAAACTGGGCTTGTCGAGACAGAGAAGACTCTTGCGTTTCTG |
| | ATAGGCACCTATTGGTCTTACTGACATCCACTTTGCCTTTCTCTCCACA |
| | GGTGTCCAGGCGGCCGCCATGGTGAGCAAGGGCGAGGAGCTGTTCACCG |
| | GGGTGGTGCCCATCCTGGTCGAGCTGGACGGCGACGTAAACGGCCACAA |
| | GTTCAGCGTGTCCGGCGAGGGCGAGGGCGATGCCACCTACGGCAAGCTG |
| | ACCCTGAAGTTCATCTGCACCACCGGCAAGCTGCCCGTGCCCTGGCCCA |
| | CCCTCGTGACCACCCTGACCTACGGCGTGCAGTGCTTCAGCCGCTACCC |
| | CGACCACATGAAGCAGCACGACTTCTTCAAGTCCGCCATGCCCGAAGGC |
| | TACGTCCAGGAGCGCACCATCTTCTTCAAGGACGACGGCAACTACAAGA |
| | CCCGCGCCGAGGTGAAGTTCGAGGGCGACACCCTGGTGAACCGCATCGA |
| | GCTGAAGGGCATCGACTTCAAGGAGGACGGCAACATCCTGGGGCACAAG |
| | CTGGAGTACAACTACAACAGCCACAACGTCTATATCATGGCCGACAAGC |
| | AGAAGAACGGCATCAAGGTGAACTTCAAGATCCGCCACAACATCGAGGA |
| | CGGCAGCGTGCAGCTCGCCGACCACTACCAGCAGAACACCCCCATCGGC |
| | GACGGCCCCGTGCTGCTGCCCGACAACCACTACCTGAGCACCCAGTCCG |
| | CCCTGAGCAAAGACCCCAACGAGAAGCGCGATCACATGGTCCTGCTGGA |
| | GTTCGTGACCGCCGCCGGGATCACTCTCGGCATGGACGAGCTGTACAAG |
| | TAATAAGCTTGGATCCAATCAACCTCTGGATTACAAAATTTGTGAAAGA |
| | TTGACTGGTATTCTTAACTATGTTGCTCCTTTTACGCTATGTGGATACG |
| | CTGCTTTAATGCCTTTGTATCATGCTATTGCTTCCCGTATGGCTTTCAT |
| | TTTCTCCTCCTTGTATAAATCCTGGTTGCTGTCTCTTTATGAGGAGTTG |
| | TGGCCCGTTGTCAGGCAACGTGGCGTGGTGTGCACTGTGTTTGCTGACG |
| | CAACCCCCACTGGTTGGGGCATTGCCACCACCTGTCAGCTCCTTTCCGG |
| | GACTTTCGCTTTCCCCCTCCCTATTGCCACGGCGGAACTCATCGCCGCC |
| | TGCCTTGCCCGCTGCTGGACAGGGGCTCGGCTGTTGGGCACTGACAATT |
| | CCGTGGTGTTGTCGGGGAAATCATCGTCCTTTCCTTGGCTGCTCGCCTG |
| | TGTTGCCACCTGGATTCTGCGCGGGACGTCCTTCTGCTACGTCCCTTCG |
| | GCCCTCAATCCAGCGGACCTTCCTTCCCGCGGCCTGCTGCCGGCTCTGC |
| | GGCCTCTTCCGCGTCTTCGAGATCTGCCTCGACTGTGCCTTCTAGTTGC |
| | CAGCCATCTGTTGTTTGCCCCTCCCCCGTGCCTTCCTTGACCCTGGAAG |
| | GTGCCACTCCCACTGTCCTTTCCTAATAAAATGAGGAAATTGCATCGCA |
| | TTGTCTGAGTAGGTGTCATTCTATTCTGGGGGGTGGGGTGGGGCAGGAC |
| | AGCAAGGGGGAGGATTGGGAAGACAATAGCAGGCATGCTGGGGACTCGA |
| | GGTGGTCCCCCGCCTCGAGTTAAGGGCGAATTCCCGATAAGGATCTTCC |
| | TAGAGCATGGCTACGTAGATAAGTAGCATGGCGGGTTAATCATTAACTA |
| | CAAGGAACCCCTAGTGATGGAGTTGGCCACTCCCTCTCTGCGCGCTCGC |
| | TCGCTCACTGAGGCCGGGCGACCAAAGGTCGCCCGACGCCCGGGCTTTG |
| | CCCGGGCGGCCTCAGTGAGCGAGCGAGCGCGCAG |
| 39 | rAAV with human CTCF multiple convergent |
| | sequence knocked in on positive-sense strand |
| | CTGCGCGCTCGCTCGCTCACTGAGGCCGCCCGGGCAAAGCCCGGGCGTC |
| | GGGCGACCTTTGGTCGCCCGGCCTCAGTGAGCGAGCGAGCGCGCAGAGA |
| | GGGAGTGGCCAACTCCATCACTAGGGGTTCCTTGTAGTTAATGATTAAC |
| | CCGCCATGCTACTTATCTACGTAGCCATGCTCTAGGAAGATCGGAATTC |
| | GCCCTTAAGCTAGCCACAAGGTGGCGCCCACAAGGTGGCGCCCACAAGG |
| | TGGCGCCCACAAGGTGGCGCCCACAAGGTGGCGCCTAGCTAGTTATTAA |
| | TAGTAATCAATTACGGGGTCATTAGTTCATAGCCCATATATGGAGTTCC |
| | GCGTTACATAACTTACGGTAAATGGCCCGCCTGGCTGACCGCCCAACGA |
| | CCCCCGCCCATTGACGTCAATAATGACGTATGTTCCCATAGTAACGCCA |
| | ATAGGGACTTTCCATTGACGTCAATGGGTGGAGTATTTACGGTAAACTG |
| | CCCACTTGGCAGTACATCAAGTGTATCATATGCCAAGTACGCCCCCTAT |
| | TGACGTCAATGACGGTAAATGGCCCGCCTGGCATTATGCCCAGTACATG |
| | ACCTTATGGGACTTTCCTACTTGGCAGTACATCTACGTATTAGTCATCG |
| | CTATTACCATGGTGATGCGGTTTTGGCAGTACATCAATGGGCGTGGATA |
| | GCGGTTTGACTCACGGGGATTTCCAAGTCTCCACCCCATTGACGTCAAT |
| | GGGAGTTTGTTTTGGCACCAAAATCAACGGGACTTTCCAAAATGTCGTA |
| | ACAACTCCGCCCCATTGACGCAAATGGGCGGTAGGCGTGTACGGTGGGA |
| | GGTCTATATAAGCAGAGCTGGTTTAGTGAACCGTCAGATCCTGCAGAAG |
| | TTGGTCGTGAGGCACTGGGCAGGTAAGTATCAAGGTTACAAGACAGGTT |
| | TAAGGAGACCAATAGAAACTGGGCTTGTCGAGACAGAGAAGACTCTTGC |
| | GTTTCTGATAGGCACCTATTGGTCTTACTGACATCCACTTTGCCTTTCT |
| | CTCCACAGGTGTCCAGGCGGCCGCCATGGTGAGCAAGGGCGAGGAGCTG |
| | TTCACCGGGGTGGTGCCCATCCTGGTCGAGCTGGACGGCGACGTAAACG |
| | GCCACAAGTTCAGCGTGTCCGGCGAGGGCGAGGGCGATGCCACCTACGG |
| | CAAGCTGACCCTGAAGTTCATCTGCACCACCGGCAAGCTGCCCGTGCCC |
| | TGGCCCACCCTCGTGACCACCCTGACCTACGGCGTGCAGTGCTTCAGCC |
| | GCTACCCCGACCACATGAAGCAGCACGACTTCTTCAAGTCCGCCATGCC |
| | CGAAGGCTACGTCCAGGAGCGCACCATCTTCTTCAAGGACGACGGCAAC |
| | TACAAGACCCGCGCCGAGGTGAAGTTCGAGGGCGACACCCTGGTGAACC |
| | GCATCGAGCTGAAGGGCATCGACTTCAAGGAGGACGGCAACATCCTGGG |
| | GCACAAGCTGGAGTACAACTACAACAGCCACAACGTCTATATCATGGCC |
| | GACAAGCAGAAGAACGGCATCAAGGTGAACTTCAAGATCCGCCACAACA |
| | TCGAGGACGGCAGCGTGCAGCTCGCCGACCACTACCAGCAGAACACCCC |
| | CATCGGCGACGGCCCCGTGCTGCTGCCCGACAACCACTACCTGAGCACC |
| | CAGTCCGCCCTGAGCAAAGACCCCAACGAGAAGCGCGATCACATGGTCC |
| | TGCTGGAGTTCGTGACCGCCGCCGGGATCACTCTCGGCATGGACGAGCT |
| | GTACAAGTAATAAGCTTGGATCCAATCAACCTCTGGATTACAAAATTTG |
| | TGAAAGATTGACTGGTATTCTTAACTATGTTGCTCCTTTTACGCTATGT |
| | GGATACGCTGCTTTAATGCCTTTGTATCATGCTATTGCTTCCCGTATGG |
| | CTTTCATTTTCTCCTCCTTGTATAAATCCTGGTTGCTGTCTCTTTATGA |
| | GGAGTTGTGGCCCGTTGTCAGGCAACGTGGCGTGGTGTGCACTGTGTTT |
| | GCTGACGCAACCCCCACTGGTTGGGGCATTGCCACCACCTGTCAGCTCC |
| | TTTCCGGGACTTTCGCTTTCCCCCTCCCTATTGCCACGGCGGAACTCAT |
| | CGCCGCCTGCCTTGCCCGCTGCTGGACAGGGGCTCGGCTGTTGGGCACT |
| | GACAATTCCGTGGTGTTGTCGGGGAAATCATCGTCCTTTCCTTGGCTGC |
| | TCGCCTGTGTTGCCACCTGGATTCTGCGCGGGACGTCCTTCTGCTACGT |
| | CCCTTCGGCCCTCAATCCAGCGGACCTTCCTTCCCGCGGCCTGCTGCCG |
| | GCTCTGCGGCCTCTTCCGCGTCTTCGAGATCTGCCTCGACTGTGCCTTC |
| | TAGTTGCCAGCCATCTGTTGTTTGCCCCTCCCCCGTGCCTTCCTTGACC |
| | CTGGAAGGTGCCACTCCCACTGTCCTTTCCTAATAAAATGAGGAAATTG |
| | CATCGCATTGTCTGAGTAGGTGTCATTCTATTCTGGGGGGTGGGGGGGG |
| | CAGGACAGCAAGGGGGAGGATTGGGAAGACAATAGCAGGCATGCTGGGG |
| | ACTCGACCGCCCCCTGGTGGCCGCCCCCTGGTGGCCGCCCCCTGGTGGC |
| | CGCCCCCTGGTGGCCGCCCCCTGGTGGTCGAGTTAAGGGCGAATTCCCG |
| | ATAAGGATCTTCCTAGAGCATGGCTACGTAGATAAGTAGCATGGCGGGT |
| | TAATCATTAACTACAAGGAACCCCTAGTGATGGAGTTGGCCACTCCCTC |
| | TCTGCGCGCTCGCTCGCTCACTGAGGCCGGGCGACCAAAGGTCGCCCGA |
| | CGCCCGGGCTTTGCCCGGGCGGCCTCAGTGAGCGAGCGAGCGCGCAG |
| 40 | rAAV with human CTCF forward on positive-sense |
| | and reverse on negative-sense strand, convergent |
| | CTGCGCGCTCGCTCGCTCACTGAGGCCGCCCGGGCAAAGCCCGGGCGTC |
| | GGGCGACCTTTGGTCGCCCGGCCTCAGTGAGCGAGCGAGCGCGCAGAGA |
| | GGGAGTGGCCAACTCCATCACTAGGGGTTCCTTGTAGTTAATGATTAAC |
| | CCGCCATGCTACTTATCTACGTAGCCATGCTCTAGGAAGATCGGAATTC |
| | GCCCTTAAGCTAGCCACAAGGTGGCGCCTAGCTAGTTATTAATAGTAAT |
| | CAATTACGGGGTCATTAGTTCATAGCCCATATATGGAGTTCCGCGTTAC |
| | ATAACTTACGGTAAATGGCCCGCCTGGCTGACCGCCCAACGACCCCCGC |
| | CCATTGACGTCAATAATGACGTATGTTCCCATAGTAACGCCAATAGGGA |
| | CTTTCCATTGACGTCAATGGGTGGAGTATTTACGGTAAACTGCCCACTT |
| | GGCAGTACATCAAGTGTATCATATGCCAAGTACGCCCCCTATTGACGTC |
| | AATGACGGTAAATGGCCCGCCTGGCATTATGCCCAGTACATGACCTTAT |
| | GGGACTTTCCTACTTGGCAGTACATCTACGTATTAGTCATCGCTATTAC |
| | CATGGTGATGCGGTTTTGGCAGTACATCAATGGGCGTGGATAGCGGTTT |
| | GACTCACGGGGATTTCCAAGTCTCCACCCCATTGACGTCAATGGGAGTT |
| | TGTTTTGGCACCAAAATCAACGGGACTTTCCAAAATGTCGTAACAACTC |
| | CGCCCCATTGACGCAAATGGGCGGTAGGCGTGTACGGTGGGAGGTCTAT |
| | ATAAGCAGAGCTGGTTTAGTGAACCGTCAGATCCTGCAGAAGTTGGTCG |
| | TGAGGCACTGGGCAGGTAAGTATCAAGGTTACAAGACAGGTTTAAGGAG |
| | ACCAATAGAAACTGGGCTTGTCGAGACAGAGAAGACTCTTGCGTTTCTG |
| | ATAGGCACCTATTGGTCTTACTGACATCCACTTTGCCTTTCTCTCCACA |
| | GGTGTCCAGGCGGCCGCCATGGTGAGCAAGGGCGAGGAGCTGTTCACCG |
| | GGGTGGTGCCCATCCTGGTCGAGCTGGACGGCGACGTAAACGGCCACAA |
| | GTTCAGCGTGTCCGGCGAGGGCGAGGGCGATGCCACCTACGGCAAGCTG |
| | ACCCTGAAGTTCATCTGCACCACCGGCAAGCTGCCCGTGCCCTGGCCCA |
| | CCCTCGTGACCACCCTGACCTACGGCGTGCAGTGCTTCAGCCGCTACCC |
| | CGACCACATGAAGCAGCACGACTTCTTCAAGTCCGCCATGCCCGAAGGC |
| | TACGTCCAGGAGCGCACCATCTTCTTCAAGGACGACGGCAACTACAAGA |
| | CCCGCGCCGAGGTGAAGTTCGAGGGCGACACCCTGGTGAACCGCATCGA |
| | GCTGAAGGGCATCGACTTCAAGGAGGACGGCAACATCCTGGGGCACAAG |
| | CTGGAGTACAACTACAACAGCCACAACGTCTATATCATGGCCGACAAGC |
| | AGAAGAACGGCATCAAGGTGAACTTCAAGATCCGCCACAACATCGAGGA |
| | CGGCAGCGTGCAGCTCGCCGACCACTACCAGCAGAACACCCCCATCGGC |
| | GACGGCCCCGTGCTGCTGCCCGACAACCACTACCTGAGCACCCAGTCCG |
| | CCCTGAGCAAAGACCCCAACGAGAAGCGCGATCACATGGTCCTGCTGGA |
| | GTTCGTGACCGCCGCCGGGATCACTCTCGGCATGGACGAGCTGTACAAG |
| | TAATAAGCTTGGATCCAATCAACCTCTGGATTACAAAATTTGTGAAAGA |
| | TTGACTGGTATTCTTAACTATGTTGCTCCTTTTACGCTATGTGGATACG |
| | CTGCTTTAATGCCTTTGTATCATGCTATTGCTTCCCGTATGGCTTTCAT |
| | TTTCTCCTCCTTGTATAAATCCTGGTTGCTGTCTCTTTATGAGGAGTTG |
| | TGGCCCGTTGTCAGGCAACGTGGCGTGGTGTGCACTGTGTTTGCTGACG |
| | CAACCCCCACTGGTTGGGGCATTGCCACCACCTGTCAGCTCCTTTCCGG |
| | GACTTTCGCTTTCCCCCTCCCTATTGCCACGGCGGAACTCATCGCCGCC |
| | TGCCTTGCCCGCTGCTGGACAGGGGCTCGGCTGTTGGGCACTGACAATT |
| | CCGTGGTGTTGTCGGGGAAATCATCGTCCTTTCCTTGGCTGCTCGCCTG |
| | TGTTGCCACCTGGATTCTGCGCGGGACGTCCTTCTGCTACGTCCCTTCG |
| | GCCCTCAATCCAGCGGACCTTCCTTCCCGCGGCCTGCTGCCGGCTCTGC |
| | GGCCTCTTCCGCGTCTTCGAGATCTGCCTCGACTGTGCCTTCTAGTTGC |
| | CAGCCATCTGTTGTTTGCCCCTCCCCCGTGCCTTCCTTGACCCTGGAAG |
| | GTGCCACTCCCACTGTCCTTTCCTAATAAAATGAGGAAATTGCATCGCA |
| | TTGTCTGAGTAGGTGTCATTCTATTCTGGGGGGTGGGGTGGGGCAGGAC |
| | AGCAAGGGGGAGGATTGGGAAGACAATAGCAGGCATGCTGGGGACTCGA |
| | GGCGGGGGGACCACCTCGAGTTAAGGGCGAATTCCCGATAAGGATCTTC |
| | CTAGAGCATGGCTACGTAGATAAGTAGCATGGCGGGTTAATCATTAACT |
| | ACAAGGAACCCCTAGTGATGGAGTTGGCCACTCCCTCTCTGCGCGCTCG |
| | CTCGCTCACTGAGGCCGGGCGACCAAAGGTCGCCCGACGCCCGGGCTTT |
| | GCCCGGGCGGCCTCAGTGAGCGAGCGAGCGCGCAG |

### REFERENCES

1. Wang D, Tai PWL, Gao G. Adeno-associated virus vector as a platform for gene therapy delivery. Nat Rev Drug Discov. 2019;18(5):358-78. doi: 10.1038/s41573-019-0012-9. PubMed PMID: 30710128; PMCID: PMC6927556.
2. Colella P, Ronzitti G, Mingozzi F. Emerging Issues in AAV-Mediated. Mol Ther Methods Clin Dev. 2018;8:87-104. Epub 20171201. doi: 10.1016/j.omtm.2017.11.007. PubMed PMID: 29326962; PMCID: PMC5758940.
3. Rao S S, Huntley M H, Durand N C, Stamenova E K, Bochkov I D, Robinson J T, Sanborn A L, Machol I, Omer A D, Lander E S, Aiden E L. A 3D map of the human genome at kilobase resolution reveals principles of chromatin looping. Cell. 2014;159(7):1665-80. Epub 2014/12/11. doi: 10.1016/j.cell.2014.11.021. PubMed PMID: 25497547; PMCID: PMC5635824.
4. Phillips-Cremins J E, Sarnia M E, Sanyal A, Gerasimova T I, Lajoie B R, Bell J S, Ong C T, Hookway T A, Guo C, Sun Y, Bland M J, Wagstaff W, Dalton S, McDevitt T C, Sen R, Dekker J, Taylor J, Corces V G. Architectural protein subclasses shape 3D organization of genomes during lineage commitment. Cell. 2013;153(6):1281-95. Epub 2013/05/23. doi: 10.1016/j.ce11.2013.04.053. PubMed PMID: 23706625; PMCID: PMC3712340.
5. Castro-Mondragon J A, Riudavets-Puig R, Rauluseviciute I, Lemma R B, Turchi L, Blanc-Mathieu R, Lucas J, Boddie P, Khan A, Manosalva Pérez N, Fornes O, Leung T Y, Aguirre A, Hammal F, Schmelter D, Baranasic D, Ballester B, Sandelin A, Lenhard B, Vandepoele K, Wasserman W W, Parcy F, Mathelier A. JASPAR 2022: the 9th release of the open-access database of transcription factor binding profiles. Nucleic Acids Res. 2022;50(D1):D165-D73. doi: 10.1093/nar/gkab1113. PubMed PMID: 34850907; PMCID: PMC8728201.
6. Boftsi M, Whittle F B, Wang J, Shepherd P, Burger L R, Kaifer K A, Lorson C L, Joshi T, Pintel D J, Majumder K. The adeno-associated virus 2 (AAV2) genome and rep 68/78 proteins interact with cellular sites of DNA damage. Hum Mol Genet. 2021. Epub 2021/10/16. doi: 10.1093/hmg/ddab300. PubMed PMID: 34652429.

What is claimed:

1. A construct for producing a recombinant adeno-associated virus (rAAV) vector, the construct comprising: a 5′ inverted terminal repeat (ITR), a first CCCTC-binding factor (CTCF) binding site, a promoter, a transgene, and a 3′ ITR.

2. The construct of claim 1 further comprising a second CTCF binding site.

3. The construct of claim 2, wherein the construct comprises from 5′ to 3′: the 5′ inverted terminal repeat (ITR), the first CCCTC-binding factor (CTCF) binding site, the promoter, the transgene, the second CTCF binding site, and the 3′ ITR.

4. The construct of claim 2, wherein the second CTCF binding site is in the convergent orientation relative to the first CTCF binding site.

5. The construct of claim 1, wherein the CTCF binding site(s) are from a human or a virus.

6. The construct of claim 5, wherein the virus is selected from the group consisting of:
adeno-associated virus (AAV), minute virus of mice (MVM), H1 parvovirus, MmuPV, B19, canine parvovirus, human cytomegalovirus (HCMV)/human herpesvirus 5 strain Merlin, human alphaherpesvirus 1, human herpesvirus 4 type 2 (Epstein-Barr virus type 2), HPV16, herpes simplex virus (HSV), and herpes B virus (HBV).

7. The construct of claim 5, wherein the CTCF binding site(s) comprise a sequence selected from: SEQ ID NOs:1-28.

8. The construct of claim 7, wherein the first CTCF binding site comprises SEQ ID NO:1 and the second CTCF binding site comprises SEQ ID NO:42.

9. The construct of claim 1, wherein the first and/or second CTCF binding site comprises multiple CTCF binding sequences.

10. The construct of claim 9, wherein the first and/or second CTCF binding site comprises five CTCF binding sequences.

11. The construct of claim 1, wherein the first CTCF binding site comprises SEQ ID NO: 3.

12. A host cell transduced with the construct of claim 1.

13. A modified rAAV virus particle comprising the construct of claim 1.

14. A packaging cell line for producing the virus particle of claim 13.

15. The packaging cell line of claim 14, wherein the cell line comprises the complement of any genes functionally deleted in the virus particle.

16. A method for producing a modified rAAV virus particle, the method comprising:
a) transducing a host cell with:
i. a plasmid comprising the construct of claim 1,
ii. a packaging plasmid, and
iii. a helper plasmid;
b) collecting the supernatant and the cells from culture; and
c) isolating virus particles from the supernatant and cells.

17. The method of claim 16 further comprising concentrating the virus particles.

18. A method of delivering a transgene to a subject, the method comprising: administering the rAAV virus particle of claim 13 to the subject.

19. The method of claim 18, wherein the transgene is expressed in a greater proportion of the subject's cells when it is delivered in the modified rAAV vector as compared to when it is delivered in a wild-type rAAV vector.

20. The method of claim 18, wherein the transgene is expressed at higher levels when it is delivered in the modified rAAV vector as compared to when it is delivered in a wild-type rAAV vector.
